(19) United States
(12) Patent Application Publication
(10) Pub. No.: US 2004/0086902 A1
(43) Pub. Date: May 6, 2004

(54) Method for the production of polyfructans

(76) Inventors: Dirk Engels, Uberlingen (DE); Alireza Haji Begli, Ramsen (DE); Markwart Kunz, Worms (DE); Ralf Mattes, Stuttgart (DE); Mohammad Munir, Kindenheim (DE); Manfred Vogel, Neuleiningen (DE)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/450,896

(22) PCT Filed: Nov. 21, 2001
(86) PCT No.: PCT/EP01/13524

(51) Int. Cl. C12Q 1/68; C07H 21/04; C12P 19/04; C08B 37/00

(52) U.S. Cl. 435/6; 435/69.1; 435/101; 435/193; 435/320.1; 435/325; 536/23.2; 536/123

(57) ABSTRACT

The present invention relates to nucleic acids encoding modified polypeptides with fructosyl transferase activity, vectors containing this nucleic acid for the expression of the modified fructosyl transferase in prokaryotic or eukaryotic cells, host cells and/or transgenic plants containing these vectors, methods for producing high-molecular polyfructans with primarily β-1,2 bonds and a very low degree of branching, in particular inulin, method for producing fructooligosaccharides using the inulin produced according to the invention or using saccharose, as well as methods for producing difructose dianhydrides using the inulin produced according to the invention or using saccharose, as well as the use of the inulin produced according to the invention for the production of inulin ethers and inulin esters, and the use of fructooligosaccharides or hydrogenated fructooligosaccharides and difructose dianhydrides as a food additive, in particular as a dietetic food.

### FIELD OF THE INVENTION

The present invention relates to nucleic acids encoding modified polypeptides with fructosyl transferase activity, vectors containing this nucleic acid for the expression of the modified fructosyl transferase in prokaryotic or eukaryotic cells, host cells and/or transgenic plants containing these vectors, methods for producing high-molecular polyfructans with primarily β-1,2-bonds and a very low degree of branching, in particular inulin, method for producing fructooligosaccharides using the inulin produced according to the invention or using saccharose, as well as methods for producing difructose dianhydrides using the inulin produced according to the invention or using saccharose, as well as the use of the inulin produced according to the invention for the production of inulin ethers and inulin esters, and the use of fructooligosaccharides or hydrogenated fructooligosaccharides and difructose dianhydrides as a food additive, in particular as a dietetic food.

### BACKGROUND OF THE INVENTION

Low-molecular saccharides, for example, monosaccharides such as glucose, and oligosaccharides such as saccharose, are used as substrates for biotechnological processes or, in modified form, as auxiliary substances in various industrial branches. For example, larger quantities of glucose are used for chemical syntheses, for example of sorbitol and glutamate, and for technical purposes, including for alcoholic fermentation. Saccharose, economically the most important type of sugar, is used primarily for food purposes and for preservation, but can also be used for plastics, varnishes, for the synthesis of protein, amino acids, antibiotics, etc., as well as an aggregate for hard PUR foams.

Polysaccharides, such as cellulose and starch, are much more frequently used in native or modified form in industry. Starch is not only the most important nutrient for mankind. Industrially obtained starch also is used in the paper industry, among other uses for producing cardboard articles or as a paper-making aid, for example for sizing paper, in the textile industry, for example as a sizing or finishing and the weighting of new fabrics or as a stiffening agent for laundry, and in the pharmaceutical industry, for example, as a disintegrant and filler for tablets, as a lubricant and filler for powders, as a base for salves, etc. Cellulose is one of the most important raw materials for many branches of industry. The largest quantities of cellulose are used in the paper and textile industry, whereby the most common textile fabrics consist, for example, of more or less pure, natural or synthetically converted cellulose. Polysaccharides or polysaccharide derivatives also are becoming more and more important as an aggregate in the construction industry.

In spite of their many established applications, both cellulose and starch have grave disadvantages. The production of chemical cellulose derivatives, i.e., of products produced, for example, via esterification and/or etherification reactions, substitutions, oxidation, or crosslinking reactions, requires, for example, the use of numerous chemical reagents, in particular the use of solvents, whose later disposal is sometimes associated with significant costs. It is therefore generally much more expensive to produce cellulose derivatives than to produce starch derivatives. Producing starch derivatives in contrast has the problem that starch is a heterogeneous compound consisting of linear-structured amylose and strongly branched amylopectin. As a result of this heterogeneity, it is not, or only to a certain extent, possible to perform a chemical starch derivatization in a reproducible and specific manner. For years, attempts therefore have been made, albeit with moderate success so far, to cultivate plants that provide exclusively amylose-containing starch. Cheap polysaccharides with a primarily linear structure that are suitable for large-scale industrial use are therefore not available on the market at this time.

For applications in which, in particular, a linear structure of the carbohydrate polymers is the crucial prerequisite for the desired property profiles, longer-chained polyfructans are an important alternative. The polyfructans are polysaccharides in which primarily fructose units are coupled with each other. Polyfructans differ from each other by the type of coupling of the fructose units. For example, the fructose units in the most important polyfructan, inulin, are present in furanoside form, with a β-1,2 bond. In the polyfructan levan, the fructose units are coupled via β-2,6 bonds. Polyfructans are reserve carbohydrates found in a number of monocotyle and dicotyle plants, for example composites, grasses, and grains, but also in algae and several gram-positive and gram-negative bacteria.

Inulin is a polyfructan with primarily linear structure, whereby fructose units are coupled via β-1,2 bonds and whereby the chain is probably terminated with a non-reducing α-D glucose unit. Inulin is found either by itself or together with starch as a reserve carbohydrate, including in dahlia tubers, artichokes, topinambur tubers, chicory roots, and in the cells of inula and other composites (compositae), more rarely also in related plant families (Campanulaceae, Lobeliaceae). Inulins from different plant families differ essentially by their average degree of polymerization. Inulin from topinambur, for example, has an average degree of polymerization of 5-7, inulin from chicory has an average degree of polymerization of 10-12, and inulin from artichokes has an average degree of polymerization of approximately 25 (Beck, R. H. F., and Praznik, W., Inulinhaltige Pflanzen aus Rohstoffquelle. Starch/Stärke, 38 (1986), 391-394). Because of their linear structure, it is relatively easy to produce derivatives from these inulins, whereby these derivatives are for the most part biologically degradable. As a result of the relatively short chains, such inulins or derivatives produced from them are not suitable for use as polymeric tensides, emulsifiers, and softeners, however. Also known is the use of inulin as a “bulking agent” or fat substitute.

It is also known to hydrogenate inulin to fructooligosaccharides and to use them as prebiotic food ingredients (Wang, X. and Gibson, G. R., J. Appl. Biochem., 75 (1993), 373-380). However, a serious disadvantage of these fructooligosaccharides is their relatively high glucose content. Fructooligosaccharides produced from the inulin of topinambur, for example, contain approximately 40 to 20% glucose, while fructooligosaccharides produced from the inulin of chicories contain approximately 8 to 10% glucose. Such fructooligosaccharides are only suitable to a limited extent for diabetic nutrition.

EP 657 106 A1 discloses the production and use of hydrogenated fructooligosaccharides. The relatively high glucose content of the fructooligosaccharides used is also a disadvantage in this case.

Of special interest are difructose dianhydrides that can also be produced from inulin and used as food ingredients. These are in particular characterized in that they develop a distinct prebiotic effect in the colon and in this way profoundly promote a healthy intestinal flora and intestinal wall. For this reason, there is great interest in the industry in a low-cost production difructose dianhydrides. Methods for producing difructose dianhydrides are known (Seki, K. et al., Starch/Stärke, 40 (1988), 440-442; DE-PS 195 47 059; Uchiyama, T., in: Science and Technology of Fructans (Ed.: Suzuki, M. and Chatterton, N.J.) (1993), CRC Boca Raton, Fla.). However, the high glucose content is also a disadvantage for the difructose dianhydrides produced in this way.

It is also known that polyfructan-containing plants, in the same manner as a number of microorganisms, express fructosyl transferase (Ftf) proteins that catalyze the polymerization of linear or branched fructose polymers. Microbial fructosyl transferases were found, for example, in Streptococcus, Bacillus, Pseudomonas, Xanthomonas, Acetobacter, Erwinia, and Actynomyces strains. In microbial polyfructans, the fructose residues are coupled via β-1,2 and/or β-2,6 bonds. This means that microbial fructosyl transferases are either inulin sucrases or levan sucrases. While plant polyfructans have a relatively low molecular weight with about 10 to 30 fructose units coupled per molecule, microbial polyfructans have a molecular weight of up to 10⁶ to 10⁸, whereby more than 100,000 fructose units may be coupled. Studies of the polyfructan biosynthesis also showed that the biosynthesis in bacteria proceeds in general substantially more simply than in plants. For these reasons, there is great interest in the use of, in particular, bacterial fructosyl transferases for producing polyfructans.

However, at the current time only one bacterial fructosyl transferase is known, i.e, the one from the gram-positive bacterium Streptococcus mutans, that is able to catalyze the formation of inulin based on saccharose as a starting molecule (Shiroza and Kuramitsu, J. Bacteriol., 170 (1988), 810-816); Rosell, K.-G. and Birkhead, D., Acta Chem. Scand., 28 (1974), 589). The inulin produced with the S. mutans fructosyl transferase from saccharose has a molar mass of 20-60×10⁶ g/mol. This means the glucose content of the inulin is negligible, so that the inulin principally would be suitable for producing fructooligosaccharides. On the other hand, inulin produced in this manner is branched approximately 7% and therefore is less suitable for use as a raw material for further chemical derivatization. The microorganism S. mutans is furthermore a human-pathogenic organism, so that its use and multiplication on an industrial scale for enzyme production is associated with major problems.

Methods for producing carbohydrate polymers, especially polyfructans, in transgenic plants using ftf genes encoding microbial fructosyl transferases, for example the fructosyl transferase gene of S. mutans, are also known.

PCT/US89/02729 describes a method for producing carbohydrate polymers, especially dextrane or polyfructose, in transgenic plants. The use of levan sucrases or dextrane sucrases of different microorganisms is suggested for producing these plants.

PCT/EP93/02110 discloses a method for producing polyfructose-producing, transgenic plants that encode the lsc gene for a levan sucrase from a gram-negative bacterium.

PCT/US94/12778 describes methods for the synthesis and accumulation of carbohydrate polymers in transgenic plants, whereby, among other things, a bacterial fructosyl transferase gene that encodes a levan sucrase is used.

PCT/NL93/00279 discloses the transformation of plants with chimeric genes that contain the sacB gene of Bacillus subtiles or the ftf gene of Streptococcus mutans. In the case of the sacB gene encoding a levan sucrase, a modification of the 5′-untranslated region of the bacterial gene is recommended in order to increase the expression level in transformed plants. No sequence modifications are described for the fructosyl transferase of Streptococcus mutans, so that the expression level of the fructosyl transferase is relatively low.

PCT/NL95/00241 describes methods for producing oligosaccharides in transgenic plants using the fructosyl transferase gene of Streptococcus mutans (Shiroza and Kuramitsu, 1988). In addition, other fructosyl transferase genes of plant origin were used. Also described was the use of the oligosaccharides produced in transgenic plants as a sugar substitute, food supplement, and bifidogenic agent in foods as well as a bifidogenic agent in animal feed.

When using the fructosyl transferase gene of in heterologous expression systems, i.e., both heterologous bacterial expression systems as well as plant systems, it was found, however, that the native protein is produced in extremely small quantities, which means that the inulin production also takes place only to a very limited extent. This small production of enzyme in heterologous host cells is associated with serious impairments of host cell growth.

### BRIEF SUMMARY OF THE INVENTION

The present invention therefore is based on the technical objective of making available methods and means, in particular methods and means based on the fructosyl transferase gene of Streptococcus mutans, for producing high-molecular polyfructans, in particular inulin, with β-1,2 bonds and an essentially linear structure and very low glucose content that enable a simple and low-cost production of the polyfructans in large quantities, whereby the previously described problems of the state of the art, in particular the low expression of bacterial fructosyl transferase genes in heterologous host systems, are overcome.

### BRIEF DESCRIPTION OF THE DRAWINGS

FIG. 1 shows a restriction map of plasmid pDHE113 that contains the complete fructosyl transferase (ftf) gene of Streptococcus mutans DSM20523 with the nucleic acid sequence of SEQ. ID No. 1 in the L-rhamnose-inducible Escherichia coli expression vector pJOE2702.

FIG. 2 shows a restriction map of plasmid pDHE225 that contains the modified fructosyl transferase gene of Streptococcus mutans DSM20523 ftf (Δ4→222) with the nucleic acid sequence of SEQ. ID No. 3 in the expression vector pJOE2702, whereby this fructosyl transferase gene is shortened at its 5′ end by 219 nucleotides.

FIG. 3 shows a restriction map of plasmid pDH171 that contains the fusion gene lacZα(1→83)::ftf(105→2388) with the nucleic acid sequence of SEQ. ID No. 5 in the expression vector pJOE2702, whereby the fusion gene comprises 83 nucleotides of the lacZα gene of vector pBluescript II KS+ and a modified fructosyl transferase gene from Streptococcus mutans DSM20523 that is shortened at the 5′ end by 104 nucleotides.

FIG. 4 shows a restriction map of plasmid pDH132 that contains the modified fructosyl transferase gene of Streptococcus mutans DSM20523 ftf (Δ2254→2385) with the nucleic acid sequence of SEQ. ID No. 7 in the expression vector pJOE2702, whereby the fructosyl transferase gene has a deletion of nucleotides 2254 to 2385.

FIG. 5 shows a restriction map of plasmid pDHE172 that contains the modified fructosyl transferase gene ftf (Δ4→222, Δ2254→2385) with the nucleic acid sequence of SEQ. ID No. 9 in the expression vector pJOE2702, whereby the fructosyl transferase gene has a deletion of nucleotides 4 to 222 and nucleotides 2254 to 2385.

FIG. 6 shows a restriction map of plasmid pDHE143 that contains the fusion gene lacZα (1→83)::ftf(105→2388, →2254→2385) with the nucleic acid sequence of SEQ. ID No. 11 in the expression vector pJOE2702, whereby the fructosyl transferase gene has a deletion of nucleotides 1 to 104 and nucleotides 2254 to 2385, and whereby the deleted 5′ region is fused to 83 nucleotides of the lacZα gene.

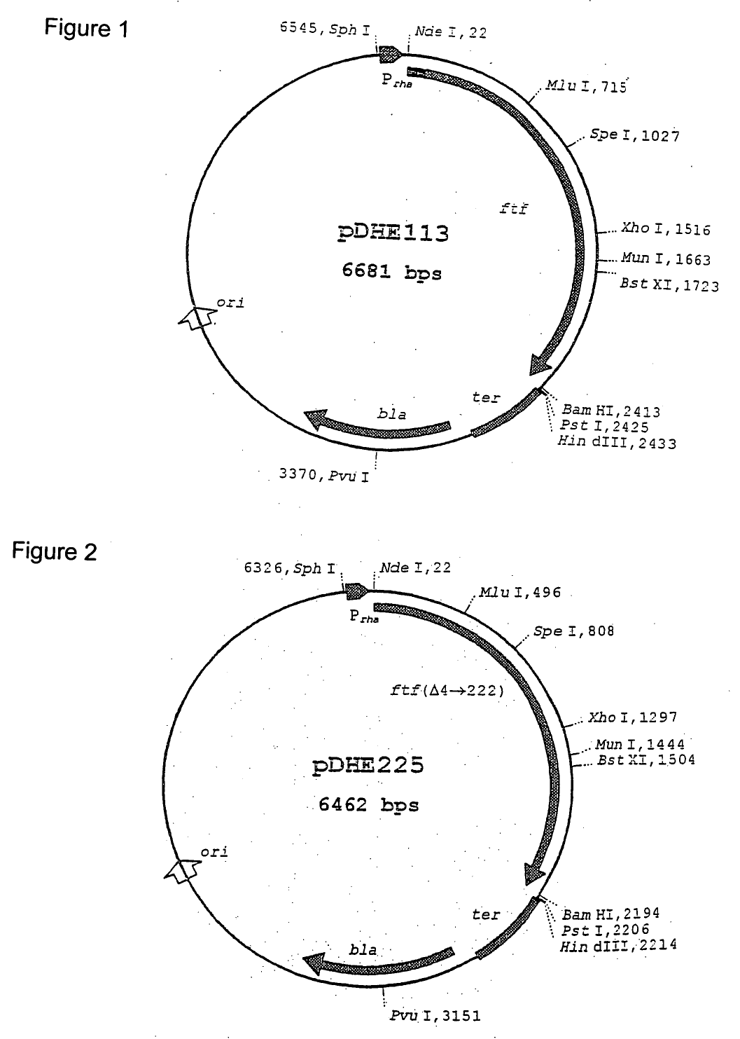

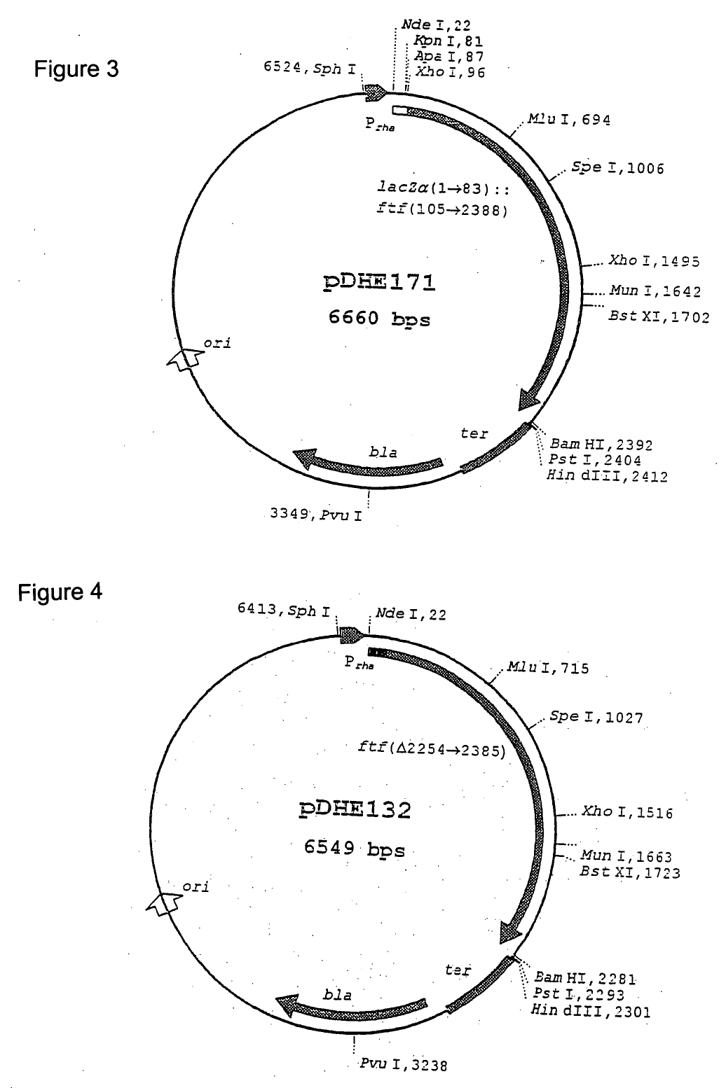

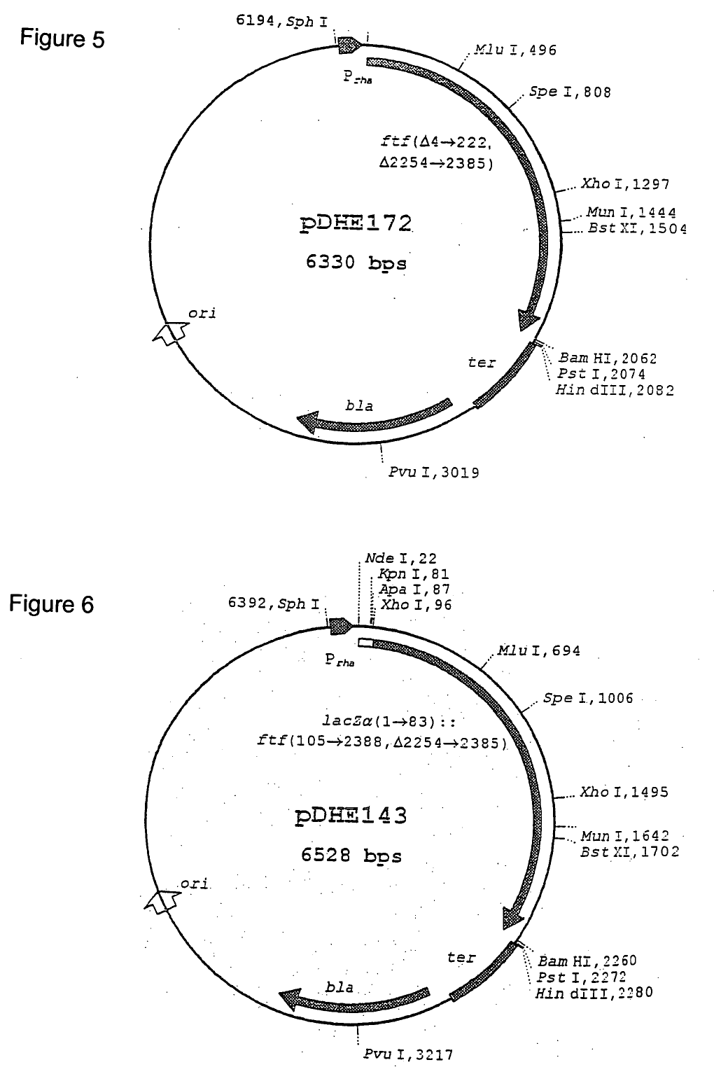

### DETAILED DESCRIPTION OF THE INVENTION

The present invention realizes the above described technical problem in particular by providing a modified fructosyl transferase gene from Streptococcus mutans with a nucleic acid sequence according to SEQ. ID No. 1 or a nucleic acid sequence encoding an amino acid sequence according to SEQ. ID No. 2, said modified fructosyl transferase gene encoding a polypeptide modified at the N terminus and/or C terminus with the activity of a fructosyl transferase, in particular whereby the polypeptide has at least one deletion at the N-terminal and/or C-terminal end. In particular, the present invention makes available a preferably isolated and completely purified nucleic acid molecule according to the principal claim, which encodes a polypeptide with the activity of a fructosyl transferase (ftf) and has at least one deletion in a nucleic acid sequence shown in SEQ. ID No. 1 or in a nucleic acid sequence encoding the amino acid sequence shown in SEQ. ID No. 2, said deletion being selected from the group consisting of:

a) Deletion of nucleotides 4 to 222;

b) Deletion of nucleotides 1 to 104; and,

c) Deletion of nucleotides 2254 to 2385.

By deleting nucleotides 4 to 222 or nucleotides 1 to 104, the signal sequence of the native fructosyl transferase gene of S. mutans is completely or partially deleted so that the encoded signal peptide of the native S. mutans fructosyl transferase is completely or partially removed. The resulting intracellular enzyme production eliminates the growth impairments of heterologous host cells, such as, for example, Escherichia coli, that are attributable to the expression of the fructosyl transferase of S. mutans, and the enzyme can be obtained in high volume yields from these cells.

It was also found unexpectedly that a deletion on the C terminus of the fructosyl transferase that comprised nucleotides 2254 to 2385 resulted in a substantial improvement in the growth of heterologous host cells. The function of the deleted hydrophobic sequence in the native protein is not known. According to the invention, this deletion within the C-terminal sequence also results in an essential improvement of the growth of heterologous host cells and high volume yields of the expressed protein.

According to the invention, it was furthermore found that the sequences deleted in the 5′ region of the nucleic acid sequence of the ftf gene can be substituted with at least one sequence region of another gene, whereby the other gene is preferably the lacZα gene. Such a mutation also brings about improved growth of heterologous host cells and high volume yields of the expressed protein.

By using the previously described nucleic acid molecules it is therefore possible to produce vectors that ensure a high expression of a modified polypeptide with the activity of a fructosyl transferase in prokaryotic and/or eukaryotic cells. According to the invention, it was determined that an especially high volume yield of the expressed protein is achieved in bacterial host systems if the nucleic acid molecules according to the invention that have deletions at the 5′ end and/or the 3′ end or the nucleic acid molecules, in which the sequences with deletions at the 5′ end have been substituted with a sequence region of another gene, are integrated into the expression cassette of the Escherichia coli vector pJOE2702 (Volff et al., Mol. Microbiol., 21 (1996), 1037-1047; Stumpp et al., Biospektrum, 1 (2000), 33-36). This vector can be induced by L-rhamnose and is positively regulated.

The prokaryotic and eukaryotic host cells produced according to the invention can be used in methods for producing polyfructans with β-1,2 bonds, whereby, after cultivation and multiplication of these host cells, a protein with the activity of a fructosyl transferase is isolated from the cells, and the isolated protein is used in vitro for treating a saccharose solution. The enzymatically produced polyfructan then can be isolated from the reaction batch and purified. In another preferred embodiment, a polyfructan can be isolated directly from host cells that contain one of the nucleic acid molecules according to the invention or from a transgenic plant that contains one of the nucleic acid molecules according to the invention. The polyfructan produced in this way is preferably inulin with a degree of polymerization of >100 and a degree of branching of ≦8%, preferably ≦3%.

The inulin produced according to the invention is used in other preferred embodiments for producing fructooligosaccharides or difructose dianhydrides. A preferred method for producing fructooligosaccharides provides that the inulin produced according to the invention is treated with an endo-inulinase, and the resulting fructooligosaccharides are then isolated from the reaction batch. Another preferred embodiment provides that a saccharose solution is treated simultaneously with a fructosyl transferase according to the invention and an endo-inulinase, and the enzymatically produced fructooligosaccharides are then isolated from the reaction solution and purified.

Other preferred embodiments of the invention relate to methods for producing difructose dianhydrides. In one embodiment, inulin produced according to the invention is treated with an endo-inulinase and cells of Arthrobacter globiformis or Arthrobacter ureafaciens. In another preferred embodiment, it is provided that a saccharose solution is treated with a fructosyl transferase according to the invention and an endo-inulinase and cells of A. globiformis or A. ureafaciens, and the resulting difructose dianhydrides are then isolated from the reaction solution and purified.

Other advantageous embodiments of the invention result from the other secondary claims.

According to the invention, a preferred embodiment also makes available a preferably isolated and purified nucleic acid molecule that is a bacterial fructosyl transferase (ftf) gene and encodes a polypeptide with the activity of a fructosyl transferase and has at least one deletion at the 5′ end and/or at the 3′ end.

In connection with the present invention, a bacterial fructosyl transferase gene or a nucleic acid molecule that encodes a bacterial polypeptide with the activity of a fructosyl transferase is understood to mean the encoding DNA sequence of a gene whose gene product has the activity of a saccharose: 2,1-β-D fructosyl transferase (EC 2.4.1.9) and that originated from a bacterium, preferably from Streptococcus mutans. In connection with the present invention, the term “fructosyl transferase” or “β-D fructosyl transferase” stands for a polypeptide or a protein that is able to catalyze the synthesis of a high-molecular carbohydrate polymer consisting of repeating fructose units, whereby saccharose serves as the starting substrate for further polymerization. In the polymer product synthesized in this manner, the repeating fructose are coupled with each other via β-1,2 bonds, so that the fructosyl transferase that catalyzes the synthesis of this product also can be called inulin sucrase. The synthesized, high-molecular polymer consisting of repeating fructose units preferably has a linear structure, may contain a terminal glucose residue originating from a saccharose molecule, and comprises at least two fructose residues. In connection with the invention, this carbohydrate is called a polyfructan. The synthesized polyfructan preferably is inulin.

The nucleic acid may be a DNA sequence, for example, part of a genomic DNA sequence, or an RNA sequence, for example an mRNA sequence or part thereof. The nucleic acid may be of natural origin, i.e., it may be isolated, for example, from Streptococcus mutans cells, or may be of synthetic origin.

The nucleic acid molecule according to the invention has in the nucleic acid sequence shown in SEQ. ID No. 1, which encodes the amino acid sequence shown in SEQ. ID No. 2, at least one deletion selected from the group consisting of a) the nucleotides 4 to 222, b) nucleotides 1 to 104, and c) nucleotides 2254 to 2385. In connection with the present invention, the term “deletion” is understood as a mutation in which part of the nucleic acid sequence present in the wild type gene is missing. Deletions can be created, for example, in vitro, using restriction enzymes if the region to be deleted is flanked by suitable restriction sites. Deletion mutations also can be introduced using certain endonucleases, for example by using the BAL-31 enzyme. Such enzymes break down the ends that were created by the restriction enzyme cleaving. However, a deletion mutagenesis also can be achieved using a mutated primer in a PCR reaction. The sequence of such a primer hereby spans the region to be deleted, whereby the primer binds to two target region sections that flank the region to be deleted. This means that the region spanned by the primer is not included in the amplification, i.e., is not amplified.

The deletions according to the invention of the fructosyl transferase gene preferably are associated with the 5′ region and the 3′ region of the native fructosyl transferase gene of Streptococcus mutans.

An especially preferred embodiment with a deletion in the 5′ regions that includes nucleotides 4 to 222 is the nucleic acid molecule with the nucleic acid sequence shown in SEQ. ID No. 3, which encodes a protein with the amino acid sequence shown in SEQ. ID No. 4. In the origin organism, S. mutans, a signal sequence mediates the secretion of the enzyme from the cytoplasm, whereby the signal sequence is cleaved during the secretion process by way of a signal peptidase. The signal peptide-dependent protein export that takes place in a similar manner in gram-positive and gram-negative bacteria, is a complex, energy-dependent process that involves a large number of soluble and membrane-bound proteins (Schatz and Beckwith, Annu. Rev. Genet., 24 (1990), 215). In the case of a desired overproduction of the fructosyl transferase protein in a heterologous host organism, the signal sequence may result in an impairment of the host's growth and thus in a reduced product yield if the signal sequence is also functional in the heterologous host and the quantity of produced protein exceeds the capacity of the secretion mechanism and/or if other physiological intracellular or extracellular processes in the cell membrane region are impaired by the recombinant protein itself or by the secretion of the recombinant protein.

A complete removal of the nucleic acid sequences encoding the signal peptide from the nucleic acid molecule with SEQ. ID No. 3 results in a complete removal of the signal peptide, so that the expression of the modified fructosyl transferase protein in a heterologous host results in an intracellular production of the gene product. As a result of the intracellular production of the fructosyl transferase, the growth impairments that occur in host systems that express the native fructosyl transferase protein, are almost completely eliminated, and the desired protein product can be obtained in high volume yields.

An especially preferred embodiment for a deletion in the 3′ region of the native fructosyl transferase gene is the nucleic acid molecule with the nucleic acid sequence shown in SEQ. ID No. 7, which nucleic acid molecule encodes a polypeptide with the amino acid sequence shown in SEQ. ID No. 8. In this nucleic acid molecule, nucleotides 2254 to 2385 have been deleted. This region has a high hydropathy index, as was shown with the help of the method by Kyte and Doolittle (J. Mol. Biol., 157 (1982), 105-142). Even though no direct function can be associated with this region, heterologous host systems show a substantially improved growth behavior during the expression of such a modified fructosyl transferase protein compared with host cells that express the native fructosyl transferase protein. The modified protein also can be isolated in high volume yields.

In another preferred embodiment, the sequences deleted in the 5′ region of the fructosyl transferase gene are substituted with the sequence region of another gene. In an especially preferred embodiment, this other gene is the lacZα gene. In connection with the present invention, the term “substitute” means that the ftf sequences encoding the signal peptide can be substituted completely or partially with equivalent sequences of another gene. When using the lacZα gene, this means that equivalent sequences of this gene are fused with the fructosyl transferase gene with deletions at its 5′ end. An especially preferred example of this is the nucleic acid molecule with the nucleic acid sequence shown in SEQ. ID No. 5, which nucleic acid molecule encodes an amino acid sequence shown in SEQ. ID No. 6. In this ftf gene variation, nucleotides 1 to 104 of the native fructosyl transferase nucleic acid sequence were substituted with nucleotides 1 to 83 of the lacZα gene. Compared with the native ftf sequence, the nucleic acid molecule has a deletion of nucleotides 1 to 104. The substitution at the 5′ end results, in the case of an expression of the fusion protein in heterologous host systems, in a localization of the gene product in the periplasmatic space. Host cells transformed with such a plasmid therefore demonstrate clearly improved growth compared with heterologous host cells containing the wild type ftf gene, and the fusion protein also can be obtained in high volume yields.

Other preferred embodiments are the nucleic acid molecule with the nucleic acid sequence shown in SEQ. ID No. 9, which nucleic acid molecule encodes an amino acid sequence shown in SEQ. ID No. 10, and the nucleic acid molecule with the nucleic acid sequence shown in SEQ. ID No. 11, which nucleic acid molecule encodes an amino acid sequence shown in SEQ. ID No. 12. The nucleic acid molecule with SEQ. ID No. 9 has at its 5′ end a deletion of nucleotides 4 to 222, and at its 3′ end a deletion of nucleotides 2254 to 2385. In the nucleic acid molecule with SEQ. ID No. 11, nucleotides 1 to 104 have been substituted with nucleotides 1 to 83 of the lacZα gene, and the 3′ end also has a deletion of nucleotides 2254 to 2385. Both ftf gene variants result in clearly improved growth in the case of expression of the corresponding gene products in heterologous host systems, whereby the corresponding gene products can be obtained in high volume yields.

The invention also comprises modified nucleic acid molecules available, for example, by substitution, addition, inversion and/or deletion of one or more bases of a nucleic acid molecule according to the invention, i.e., also nucleic acid molecules that that may be called mutants, derivatives, and functional equivalents, i.e., structurally different, but functionally identical or functionally similar variations of a nucleic acid molecule according to the invention. The sequence of nucleic acid molecules, for example, may be specifically further modified in order to create suitable restriction sites within the nucleotide sequence or to remove unnecessary sequence parts. The modifications of the nucleic acid molecules according to the invention may be performed using standard microbiology/molecular biology methods. For this purpose, the respective nucleic acids are inserted into plasmids and are subjected to mutagenesis methods or sequence modification by recombination. For example, methods for in vitro mutagenesis, primer repair methods, as well as restriction and/or ligation methods are suited to bring about insertions, deletions, or substitutions, such as transitions or transversions (cf. Sambrook et al., 1989, Molecular Cloning: A Laboratory Manual, 2nd edition (1989), Cold Spring Harbor Laboratory, Cold Spring Harbor, N.Y., USA). Other sequence modifications also may be achieved by the attachment of natural or synthetic nucleic acid sequences.

The present invention furthermore relates to vectors that contain the nucleic acid molecules according to the invention. These vectors are preferably plasmids, cosmids, viruses, liposomes, bacteriophages, shuttle vectors, and other vectors usually employed in genetic engineering. The vectors according to the invention may contain other functional elements that bring about or at least contribute to a stabilization and/or replication of the vector in a host cell.

In an especially preferred embodiment, the present invention comprises vectors in which at least one nucleic acid molecule according to the invention is under the functional control of at least one regulatory element. According to the invention, the term “regulatory element” means such elements that ensure the transcription and/or translation of nucleic acid molecules in prokaryotic and/or eukaryotic host cells, so that a polypeptide or protein is expressed. The regulatory elements may be promoters, enhancers, silencers, and/or transcription termination signals. Regulatory elements that are functionally connected with a nucleotide sequence according to the invention, in particular the protein-encoding sections of this nucleotide sequence, may be nucleotide sequences originating from other organisms or other genes than the protein-encoding nucleotide sequence itself. Example for this are: T7, T3, SP6, and other commonly used regulatory elements for in vitro transcription; P_{LAC}, P_{Ltet}, and other commonly used regulatory elements in E. coli; GAL1-10, MET25, CUP1, ADH1, AFH1, GDH1, TEF1, PMA1, and other regulatory elements for expression in baker's yeast S. cerevisiae; polyhedrin for expression in Baculovirus systems; P_{CMV}, P_{SV40}, and other commonly used regulator elements for expression in mammalian cells; tissue- or organ-specific, in particular storage-organ-specific promoters, e.g., the vicillin promoter of Pisum sativum, the Arabidopsis promoter AtAAP1, or the patatin B33 promoter, for the expression in plant systems.

In another embodiment, the invention provides that the nucleic acid molecules according to the invention are fused with a signal sequence that encodes a signal peptide for inclusion into the endoplasmatic reticulum of a plant cell and for further transport into the vacuole. A vacuolar localization of the gene products is especially advantageous. According to the invention, for example, signal peptides for the vacuolar localization of lectin from barley, signal sequences of a patatin gene of the potato, or signal sequences of mature phytohemagglutinin of the bean may be used.

A preferred embodiment provides that the regulatory elements stem from the L-rhamnose operon of Escherichia coli. In an especially preferred embodiment, a nucleic acid molecule according to the invention is integrated into the expression cassette of the pJOE2702 vector (Volff et al., 1996; Stumpp et al., 2000). The expression cassette comprises the rhaₚ promoter from the L-rhamnose operon rhaBAD of Escherichia coli that is regulated at two levels (Egan and Schleif, J. Mol. Biol., 243 (1994), 821-829). This means that the pJOE2702 vector is an L-rhamnose-inducible expression vector that can be positively regulated at two levels and is present in a high number of copies in the Escherichia coli host bacterium. The transcription termination and translation initiation of the transcripts also take place via sequences of the expression cassette contained in the vector. pJOE2702 has two critical advantages over most commercially available E. coli expression vectors. Firstly, in the non-induced state, the vector leads to a very low base expression of the polypeptide to be expressed. Secondly, the transcription of the expression cassette is induced with a delay. The vector is therefore especially suitable for cloning and producing proteins that negatively influence the vitality and growth properties of the host cell, such as, for example, of bacterial fructosyl transferases. Even though Escherichia coli cells containing the pJOE2702 vector with the complete S. mutans fructosyl transferase gene show complete inhibition of growth after induction, the vector is especially suitable for expressing the nucleic acid molecules according to the invention that contain at least one of the deletions according to the invention at the 5′ and 3′ end.

The present invention naturally also comprises vectors that contain not only one but several of the nucleic acid molecules according to the invention. The nucleotide sequences hereby can be arranged in such a way that, as applicable, one, two, or more of the nucleotide sequences, in particular of the sequences described in SEQ. ID No. 3, SEQ. ID No. 5, SEQ. ID No. 7, SEQ. ID No. 9, or SEQ. ID No. 11, are controlled by a single set of regulator elements.

In a preferred embodiment, the present invention relates to host cells that include one or more of the nucleic acid molecules according to the invention or one or more of the vectors according to the invention, and which are able to express the polypeptides that are encoded by the nucleic acid molecules and have the activity of a fructosyl transferase. The host cells according to the invention also may be both prokaryotic as well as eukaryotic cells. Preferred examples of prokaryotic cells are bacteria, such as, for example, Escherichia coli or Bacillus subtilis. Examples of eukaryotic host cells that are preferred according to the invention include yeast cells, insect cells, and plant cells. The host cell according to the invention may be characterized in that the nucleotide sequence according to the invention that was introduced is heterologous in relation to the transformed cell, i.e., the nucleotide sequence according to the invention that was introduced does not naturally occur at this location or is located at a different location in these cells or in a different number of copies or a different orientation in the genome of these cells than the corresponding, naturally occurring sequence.

In an especially advantageous embodiment of the present invention, the host cell is a gram-negative cell, in particular an Escherichia coli cell. In another advantageous embodiment, it may also be a gram-positive cell, for example a Bacillus subtilis cell.

In another preferred embodiment, the host cell according to the invention is a eukaryotic host cell. In an especially preferred embodiment, the host cell according to the invention may be a yeast cell, for example a Saccharomyces cerevisiae cell. Other preferred cells include insect cells, for example, the insect cell line IPLB-Sf21. In an especially preferred embodiment, the host cell is a plant cell, in particular a cell of such plants that naturally produce polyfructans, in particular inulin, such as, for example a topinambur, artichoke, or chicory cell, or cells of agriculturally important plants that naturally produce other monosaccharides, oligosaccharides, and/or polysaccharides, for example a potato, manioc, or sugar beet cell.

The invention also relates to cell cultures with at least one of the host cells according to the invention, whereby a cell line according to the invention has the ability of producing a polypeptide or protein with the activity of a fructosyl transferase or a fragment thereof.

The invention also relates to plants that contain in at least one of their cells at least one nucleic acid molecule according to the invention or at least one vector according to the invention, or which contain at least one, but preferably a plurality, of host cells with the fructosyl transferase gene according to the invention or vectors or plasmids containing it, and which as a result are able to produce high-molecular polyfructans, in particular high-molecular inulin. The invention also makes it possible to make available plants of very different species, genus, families, orders, and classes, which as a result of the introduced nucleic acid molecules are able to produce high-molecular inulin. In contrast to the few plants that are able to produce inulin naturally, this has the advantage of enabling a specific localization of the formed inulin and that additionally an increase in the expression rate and thus in the quantity of the inulin formed is achieved. The inulin produced in transgenic plants also has a higher molecular weight than naturally produced plant inulin. While naturally produced plant inulin has on average 10 to 30 fructose units per molecule, the polyfructan formed in transgenic plants may have more than 100,000 fructose units.

The invention also relates to a transgenic harvesting and multiplication material of the plant that contains a nucleic acid molecule according to the invention, as well as parts or calli of a plant according to the invention, for example storage organs, fruit, tubers, beets, seeds, leaves, flowers, etc.

The invention provides, in particular, that the plant to be transformed is either a plant that is able to naturally produce a polyfructan, in particular inulin, preferably a topinambur, artichoke, or chicory plant, or an agriculturally important plant that is able to naturally produce monosaccharides, oligosaccharides, or polysaccharides, preferably a potato, manioc, or sugar beet plant.

The invention also relates to methods for producing the previously mentioned plants, comprising the transformation of one or more plant cells with a vector according to the invention, the integration of the nucleic acid molecule contained in this vector into the genome of the plant cell(s), and the regeneration of the plant cell(s) into intact, transformed plants that are able to produce a high-molecular polyfructan, in particular inulin.

The invention also relates to a modified polypeptide or protein with the activity of a fructosyl transferase that is able to catalyze the transformation of saccharose into a polyfructan, in particular inulin, with furanoside β-1,2 bonds. The present invention relates in particular a preferably isolated and completely purified protein that can be obtained by expression of a nucleic acid molecule according to the invention or a fragment thereof in a host cell according to the invention or a plant according to the invention, and which has the previously mentioned biological activity. The protein preferably has the same properties, in particular the same activity of a fructosyl transferase, as the protein encoded by a nucleic acid molecule with the nucleotide sequence shown in SEQ. ID No. 1 and whose amino acid sequence is shown in SEQ. ID No. 2.

The present invention also includes isolated and completely purified monoclonal or polyclonal antibodies or their fragments, which react with a polypeptide or protein according to the invention so specifically and with such an affinity that the use of these monoclonal or polyclonal antibodies or their fragments, with the help of the usual immunological methods, makes it possible, for example, to identify a protein according to the invention. A preferred embodiment of the invention therefore comprises monoclonal and polyclonal antibodies that are able to specifically identify a structure of a polypeptide or protein according to the invention with the activity of a fructosyl transferase and/or may bind to it. Such a structure may be a protein, peptide, carbohydrate, proteoglycane, and/or a lipid complex that is part of the protein according to the invention or is a specific relationship with it. The invention also comprises antibodies against structures that were created as a result of post-translational modifications of the protein according to the invention. The invention also comprises fragments of such antibodies, for example Fc or F(ab′)₂ or Fab fragments.

The present invention also relates to antibodies against an antibody according to the invention, i.e., are able to identify an antibody according to the invention and bind to it, enabling a specific identification of the antibodies according to the invention.

The present invention furthermore relates to methods for producing longer-chained polyfructans with linear structure, whereby the fructose units in the chain are coupled by β-1,2 bonds, and with a degree of polymerization of more than 100 and a degree of branching of less than 3%. In an advantageous embodiment, it is provided that the polyfructan is isolated from transgenic plants that were transformed according to the invention, in particular from their vacuoles, and is purified. Especially preferred is the isolation and obtaining of polyfructans from the storage organs of potato, topinambur, artichoke, chicory, manioc, or sugar beet plants. The method comprises the mechanical comminuting of larger plant cell masses by using a turbine mixer, for example, a Waring Blender, whereby the comminuting preferably takes place in a large volume of aqueous medium at low temperatures, preferably at +4° C. Further breakdown may be performed with the help of physical breakdown methods, for example by using ultrasound, a “French press” device, mills, or presses, with the help of chemical breakdown methods, for example a lyophilization, or by using detergents or by using osmotic pressure change, or with the help of biological breakdown methods, for example by using enzymes attacking the cell wall or by an acid/base treatment. In an especially preferred embodiment, the high-molecular polyfructan is obtained by preparation of an aqueous extract, in particular of the storage organs, and subsequent precipitation with alcohol.

Another advantageous embodiment of the method for producing polyfructans provides that host cells according to the invention, for example plant host cells or microbial host cells, for example Escherichia coli cells, are cultivated and multiplied in a suitable nutrient medium and under suitable cultivation conditions. The cell material produced is then broken down with the help of suitable physical, chemical, and/or enzymatic methods, and the proteins with the activity of a fructosyl transferase are purified from the broken down material. The further purification of enzymatic activity takes place with the help of standard methods known in this field. To obtain a raw extract, the breakdown solution is treated, for example, with extraction, centrifugation, and filtration processes. The precipitation of the proteins from the raw extract as well as ultrafiltration processes are efficient methods for concentrating proteins from a larger volume of fluid. Precipitation agents that are used include, among others, inorganic salts, for example sodium and ammonium sulfate, organic solvents, for example alcohols, and polymers, for example polyethylene glycol. To separate the precipitation agents used, a subsequent dialysis process may be performed. Another fine purification may be performed using chromatographic methods and distribution methods, for example by using aqueous phase systems. These methods include, among others, adsorption chromatography, ion exchange chromatography, gel chromatography, and affinity chromatography.

As the case may be, the isolated fructosyl transferase also can be immobilized using adsorption on an inert or electrically charged, inorganic, or organic carrier medium. Carrier materials used are inorganic materials, such as porous glasses, silica gel, aluminum oxide, hydroxyapatite, or various metal oxides, natural polymers, for example cellulose, starch, alginate, agarose, or collagen, or synthetic polymers, such as polyacrylamide, polyvinyl alcohol, methylacrylate, nylon, or oxiranes. The immobilization is hereby accomplished by physical binding forces, such as, for example, van der Waals forces, hydrophobic interactions, and ionic bonds. The immobilization of the fructosyl transferase also may take place using a covalent bond with carrier materials. For this purpose, the carriers must have reactive groups that are able to form homopolar bonds with amino acid side chains. Suitable groups include, for example, carboxy, hydroxy, and sulfide groups. The surface of porous glasses can be activated, for example by treatment with silanes, and then can be converted with proteins. Hydroxy groups of natural polymers can be activated with bromocyane and carboxy groups with thionyl chloride and then coupled with enzymes. Another possibility for immobilizing fructosyl transferases is their integration in three-dimensional networks. One advantage of this is that the enzymes are present in the network in free, unbound form. The pores of the surrounding matrix must be small enough hereby to hold back the enzyme.

After the fructosyl transferase has been obtained from the host cells according to the invention, either as a raw extract, in highly purified form, and/or in immobilized form, on a solid carrier, a saccharose solution is treated under suitable conditions with the isolated enzyme, whereby a polyfructan is formed in vitro. Suitable conditions hereby include a suitable temperature, preferably 30° C., a suitable pH value, a suitable buffer, and suitable substrate concentrations. The polyfructan formed is then isolated from the reaction batch and is purified.

The invention also relates to the high-molecular polyfructan produced with the help of the previously mentioned methods. The polyfructan produced in this way is characterized by a linear structure, whereby fructose units are coupled within the structure via β-1,2 bonds, and whereby the chain carries a non-reducing α-D glucose unit as a termination. The polyfructan is characterized by a very high degree of polymerization of >100 and a very low degree of branching of <3%. Because of the low degree of branching, the polyfructan prepared in this way has only few terminal glucose units. The polyfructan is preferably inulin, which is characterized by a high molecular weight of more than 1.5 million Dalton.

Because of the low glucose content, the polyfructan, in particular inulin, produced according to the invention can be used to produce fructooligosaccharides that are suitable as a dietetic food.

A preferred embodiment of the invention therefore also relates to methods for producing fructooligosaccharides. In an especially preferred embodiment of the invention, the inulin produced according to the invention is treated under suitable conditions with an immobilized or non-immobilized, suitable endo-inulinase, and the produced fructooligosaccharides are then isolated from the reaction batch and are purified. According to the invention, an “endo-inulinase” means a 2,1-β-D fructan fructan hydrolase that catalyzes the breakdown of inulin to fructooligosaccharides, whereby the enzymatic effect acts in particular within the polymer chain. In connection with the present invention, “fructooligosaccharides” are saccharides that consist of 2 to 10 fructose units that are bound to each other β-glycosidically. The used endo-inulinase hereby can be present in immobilized or non-immobilized form. The immobilization of the endo-inulinase hereby may take place in the way described above for the fructosyl transferase according to the invention. Suitable conditions hereby include a suitable temperature, preferably 30° C., a suitable pH value, a suitable buffer, and suitable substrate concentrations.

In another preferred embodiment of the method for producing fructooligosaccharides it is provided that a saccharose solution is treated in vitro under suitable conditions simultaneously with an immobilized or non-immobilized fructosyl transferase produced according to the invention and an immobilized or non-immobilized endo-inulinase, and the produced fructooligosaccharides are subsequently isolated from the in vitro reaction batch and are purified.

The invention therefore also relates to fructooligosaccharides that have been produced according to one of the previously mentioned methods. The fructooligosaccharides produced with the help of the method according to the invention are characterized in particular by a very low glucose content and are therefore especially suitable as a dietetic food, especially for diabetics. While fructooligosaccharides produced from inulin from topinambur have a glucose content of approximately 14 to 20%, and fructooligosaccharides produced from inulin of chicories have a glucose content of approximately 10%, the fructooligosaccharides produced according to the invention have a glucose content of less than 3%, preferably of less than 1%.

The fructooligosaccharides produced according to the invention also may be subjected to hydrogenation, whereby hydrogenated fructooligosaccharides are produced that are also especially suitable as a dietetic food. The hydrogenation of the fructooligosaccharides produced according to the invention may be performed, for example, by using higher pressures and by using catalyzers.

Finally, the present invention relates to methods for producing difructose dianhydrides. Difructose dianhydrides are of special interest as a food additive, since they are indigestible in the small intestines and develop a distinctly prebiotic effect in the colon, and in this way profoundly promote a healthy intestinal flora and intestinal wall.

One embodiment of the invention provides that inulin produced according to the invention is treated under suitable conditions simultaneously with an immobilized or non-immobilized endo-inulinase and immobilized or non-immobilized cells of Arthrobacter globiformis or Arthrobacter ureafaciens. As described above, the endo-inulinase hereby catalyzes the conversion of inulin into fructooligosaccharides. These are then transformed by the inulin fructotransferase of A. globiformis or A. ureafaciens (Seki et al., Starch/Stärke, 40 (1988), 440-442). According to one advantageous embodiment, the cells of A. globiformis or A. ureafaciens are present in immobilized form. To immobilize the microorganisms, cultivated, inactivated cells can be used directly as biocatalyzers after suitable copolymerization, for example with addition of a neutral protein and cross-linking with glutardialdehyde. It is also possible to enclose vital microorganisms in polymers, for example carrageen, and then incubate in a suitable nutrient medium. The polymer particles then can be used for the actual process and even may be regenerated, as needed, by repeat incubation. It is also possible to use microorganisms in the form of photocrosslinked polymers, whereby a microorganism suspension is polymerized with the soluble prepolymers in a thin layer by irradiation with, for example, a daylight lamp.

Suitable conditions for producing difructose dianhydrides hereby include a suitable temperature, preferably 30° C., a suitable pH value, a suitable buffer, and suitable substrate concentrations.

Another preferred embodiment of the method for producing difructose dianhydrides provides that a saccharose solution is treated simultaneously with an immobilized or non-immobilized fructosyl transferase according to the invention, an immobilized or non-immobilized endo-inulinase, and immobilized or non-immobilized cells of Arthrobacter globiformis or Arthrobacter ureafaciens, and the difructose dianhydrides produced are then isolated from the reaction batch and are purified.

Further embodiments of the invention provide for the use of the inulin produced according to the invention in different non-food applications. The inulin produced according to the invention is subjected to corresponding derivatization reactions, whereby inulin ethers and inulin esters are produced. The inulin ethers and inulin esters produced in this way can be used, for example, as polymeric tensides, softeners, or emulsifiers.

The present invention furthermore relates to the use of the fructooligosaccharides produced according to the invention, the hydrogenated fructooligosaccharides produced according to the invention, and the difructose dianhydrides produced according to the invention as a food additive, dietetic food, and animal feed additive.

The sequence protocol for the teaching according to the invention includes the following:

SEQ. ID No. 1 shows the DNA sequence (comprising 2388 nucleotides) of the ftf gene of Streptococcus mutans DSM20523.

SEQ. ID No. 2 shows the amino acid sequence (comprising 795 amino acids) of the fructosyl transferase protein according to the invention, derived from SEQ. ID No. 1.

SEQ. ID No. 3 shows the DNA sequence (comprising 2169 nucleotides) of the modified fructosyl transferase gene ftf (Δ4→222).

SEQ. ID No. 4 shows the amino acid sequence (comprising 722 amino acids) of a modified fructosyl transferase protein, derived from SEQ. ID No. 3.

SEQ. ID No. 5 shows the DNA sequence (comprising 2367 nucleotides) of the fusion gene lacZα(1→83)::ftf(105→2388).

SEQ. ID No. 6 shows the amino acid sequence (comprising 788 amino acids) of a fusion protein according to the invention, derived from SEQ. ID No. 5.

SEQ. ID No. 7 shows the DNA sequence (comprising 2256 nucleotides) of the modified fructosyl transferase gene ftf (Δ2254→2385).

SEQ. ID No. 8 shows the amino acid sequence (comprising 751 amino acids) of a fructosyl transferase protein modified to the invention, derived from SEQ. ID No. 7.

SEQ. ID No. 9 shows the DNA sequence (comprising 2037 nucleotides) of the modified fructosyl transferase gene ftf (Δ4→222, Δ2254→2385) according to the invention.

SEQ. ID No. 10 shows the amino acid sequence (comprising 678 amino acids) of a fructosyl transferase protein modified according to the invention, derived from SEQ. ID No. 9.

SEQ. ID No. 11 shows the DNA sequence (comprising 2235 nucleotides) of the fusion gene lacZα(1→83)::ftf(105→2388, Δ2254→2385) according to the invention.

SEQ. ID No. 12 shows the amino acid sequence (comprising 744 amino acids) of a fusion protein according to the invention, derived from SEQ. ID No. 11.

SEQ. ID No. 13 shows the sequence of primer ftf1(upper).

SEQ. ID No. 14 shows the sequence of primer ftf2(lower).

SEQ. ID No. 15 shows the sequence of primer ftf3(upper).

SEQ. ID No. 16 shows the sequence of primer ftf4(upper).

SEQ. ID No. 17 shows the sequence of primer ftf5(upper).

SEQ. ID No. 18 shows the sequence of primer ftf6(lower).

The following examples are intended to explain the invention in more detail, but are not intended to limit the scope of protection of the invention.

### EXAMPLE 1

### Cloning of the ftf Gene of Streptococcus mutans DSM20523

In order to clone the fructosyl transferase gene of S. mutans DSM20523, a polymerase chain reaction (PCR) was performed, whereby the primer ftf1(upper) with the sequence 5′TATATATCATATGGAAACTAAAGTTAG-3′, and the primer ftf2(lower) with the sequence 5′-TAGGATCCTTATTTAAAACCAATGCT-3′ were used. The primer ftf1(upper) hereby contained a NdeI restriction site, and the primer ftf2(lower) contained a BamHI restriction site. Chromosomal DNA of S. mutans DSM20523, which had been isolated with the help of a commercially available purification kit, was used as a template in the PCR reaction. The PCR reaction was performed in 40 μl reaction volume with 8 pmol each primer, 50 ng DNA template, and 2.5 units Pwo polymerase in 10 mM Tris-HCl, pH value 8.85, 25 mM KCl, 5 mM (NH₄)SO₄, 2 mM MgSO₄, 5% dimethyl sulfoxide, 0.2 mM dATP, 0.2 mM dTTP, 0.2 mM dGTP, and 0.2 mM dCTP. The PCR reaction hereby comprised the following steps: 5 minutes of denaturation at 96° C., 5 cycles, each comprising a 1 minute denaturation at 63° C., a 30 second primer addition at 40° C., and a 2 minute 30 second polymerization at 68° C., 25 further cycles, each comprising a 1 minute 30 second primer addition at 92° C., a 1 minute 30 second primer addition at 49° C. and a 2 minute 30 second polymerization at 68° C., as well as a 5 minute end polymerization reaction at 68° C. By using the PCR reaction, a fragment with a size of approximately 2.4 kb was amplified. The isolated fragment was cleaved with restrictases NdeI and BamHI and was ligated with the vector pJOE2702 (Volff et al., Mol. Microbiol., 21 (1996) 1037-1047; Stumpp et al., Biospectrum, 1 (2000) 33-36) that had been cleaved with the same restrictases. The encoding sequence of the ftf gene therefore was cloned in the correct reading frame into the L-rhamnose-inducible expression cassette contained in the vector, so that the transcription of the inserted nucleic acid is under the control of the rhap promoter contained in vector pJOE2702. The transcription termination of the ftf gene and the translation initiation of the transcripts also were performed via sequences of the vector. The E. coli strain JM109 then was transformed with the plasmid pDHE113 resulting from ligation. Transformants were hereby selected by way of ampicillin resistance.

### EXAMPLE 2

### Modifications of the Cloned ftf Gene From Streptococcus mutans DSM20523

In order to modify the ftf gene product in the region of the N-terminal signal peptide, deletions were introduced or sequence regions were substituted at the 5′-OH end of the ftf gene.

In order to produce the modified fructosyl transferase gene ftf (Δ4→222), a PCR reaction was performed with the primer ftf3(upper) with the sequence 5′-TATATATCATATGGAAACTCCATCAACAAATCCCG-3′ and the primer ftf2(lower). The primer ftf3(upper) contains a NdeI site. Purified DNA of plasmid pDHE113 that contains the cloned, complete ftf gene of S. mutans DSM20523 was used as a template. The PCR reaction was performed in 40 μl reaction volume with 8 pmol each primer, 100 ng plasmid DNA template, and 2.5 units Pwo polymerase in 10 mM Tris-HCl, pH value 8.85, 25 mM KCl, 5 mM (NH₄)SO₄, 2 mM MgSO₄, 0.2 mM dATP, 0.2 mM dTTP, 0.2 mM dGTP, and 0.2 mM dCTP. The PCR reaction comprised the following steps: 2 minutes of denaturation at 94° C., 25 cycles, each comprising a 1 minute denaturation at 93° C., a 1 minute 30 second primer addition at 55° C., and a 2 minute 20 second polymerization at 68° C., as well as a 5 minute end polymerization at 68° C. The obtained, approximately 2.2 kb PCR product with the activity of a fructosyl transferase isolated, cleaved with NdeI and BamHI, and ligated into the vector pJOE2702 that had been cleaved with the same restriction enzymes. The E. coli strain JM109 was then transformed with plasmid pDHE225 that resulted from the ligation. The gene product of the ftf(Δ1→222) variant is 73 amino acids shorter than the wild type sequence at its N terminus.

In order to produce the modified fusion gene lacZα(1→83)::ftf(105→2388), a PCR reaction was performed with the primer ftf4(upper) with the sequence 5′ATATATGTCGACGGCAGATGAAGCCAATTCAAC-3′ and the primer ftf2(lower). The primer ftf4(upper) contains a SalI site. A purified DNA of plasmid pDHE113 that contains an insertion of the complete gene of S. mutans DSM20523 was used as a template. The PCR reaction was performed as described above. The resulting, approximately 2.3 kb PCR product was isolated, cleaved with restrictases SalI and BamHI, and cloned into the vector pBluescript II KS+, whereby the shortened ftf reading frame at the 5′ end was fused with the beginning region of the lacZα reading frame contained in the vector. The E. coli strain JM109 was then transformed with the resulting plasmid pDHE166. In a second PCR step, the fusion gene lacZα(1→83)::ftf(105→2388) contained in the vector pDHE166 was recloned into the expression vector pJOE2702. For this purpose, a PCR reaction was performed with the primer ftf5(upper) with the sequence 5′TATATATCATATGACCATGATTACGCCAAGC-3′ and the primer ftf2(lower). The primer ftf5(upper) has a sit for restrictase NdeI. A purified DNA of plasmid pDHE166 was used as a template. The PCR reaction was performed as described above. The resulting, approximately 2.4 kb PCR product was isolated, cleaved with restrictases NdeI and BamHI, and ligated into the vector pJOE2702 that had been cleaved with the same restrictases. The E. coli strain JM109 was then transformed with plasmid pDHE171 resulting from ligation.

To produce the modified fructosyl transferase gene ftf(Δ2254→2385), the PCR primer ftf6(lower) with the sequence 5′TTGGATCCTTATTTTGAGAAGGTTTGACAG-3′ was used in combination with others of the previously described primers. The primer ftf6(lower) contains a site for restrictase BamHI. The purified DNA of plasmid pDHE113 was used as a template. The PCR reaction was performed as described above. Then the amplification product was isolated, cleaved with restrictases NdeI and BamHI, and ligated into vector pJOE2702 that had been previously cleaved with the same restrictases. By using the primer combination ftf1(upper)/ftf6(lower), the plasmid pDHE132 that contains the previously described, modified ftf gene was obtained.

To produce the modified fructosyl transferase gene ftf(Δ4→222, Δ2254→2385), a PCR reaction was performed using the DNA pf plasmid pDHE113 as a template and primers ftf3(upper) and ftf6(lower). After integration of the amplification product into vector pJOE2702, the plasmid pDHE172 was obtained.

To produce the modified fructosyl transferase gene lcZα(1→83)::ftf(105→2388, Δ2254→2385), a PCR reaction was performed using the DNA of vector pDHE113 and primers ftf4(upper) and ftf6(lower). The resulting, approximately 2.2 kb PCR product was integrated via sites SalI ad BamHI into the vector Bluescript II KS+, resulting in the plasmid pDHE140. Then a second PCR reaction was performed, whereby the primers ftf5(upper) and ftf6(lower) as well as the DNA of plasmid pDHE140 were used as a template. The resulting, approximately 2.3 kb PCR product was isolated, cleaved with restrictases NdeI and BamHI, and ligated into vector pJOE2702 that had been previously cleaved with the same restrictases. This yielded the plasmid pDHE143.

### EXAMPLE 3

### Heterologous Expression of the Different Variants of the ftf Gene in E. coli

In order to detect the gene products in raw extracts of the E. coli strain JM109 used for expression, E. coli strains that contained one of the previously produced plasmids or the plasmid pJOE2702 for control were cultivated and induced. The strains were precultivated overnight in 5 ml dYT full medium with 100 μg of ampicillin/ml at 30° C. in the incubation roller (Sambrook et al., Molecular Cloning: A Laboratory Manual, 2nd Edition (1989), CSH Laboratory Press, Cold Spring Harbor, N.Y.). Using this overnight culture, 50 ml each of dYT medium were inoculated in such a way that the optical density at 600 nm (OD₆₀₀) was approximately 0.02. The cells were then cultivated in Erlenmeyer flasks on the incubation agitator to an OD₆₀₀ of 0.2. To induce the producer inducible with L-rhamnose, 0.2% (weight/Volume) of L-rhamnose was added to the medium. Then the cells were incubated for another 7 hours. After 7 hours of incubation, the cells were harvested by centrifugation, washing of the cells in 50 mM KH₂PO₄/K₂HPO₄ buffer, ph value 6.5, and subsequent resuspension of the cells in the same buffer. The suspension was then adjusted to an OD₆₀₀ value of 10. The cells were broken down using ultrasound treatment or French press treatment. All extracts were then treated by 20 minutes of centrifugation at 10,000×g. Then the total protein content was determined according to Bradford (Bradford, Anal. Biochem., 72 (1976), 248-254), whereby a beef serum albumin-calibrated reagent (Biorad-Laboratories GmbH, Munich) was used. To compare the protein patterns of the resulting raw extracts with the protein pattern of the non-induced control, the extracts were separated electrophoretically in 7.5% SDS polyacrylamide gels (Laemmli, Nature, 227 (1997) 680-685).

The Ftf activities were determined quantitatively by measuring the specific Ftf activities in raw extracts. Cell cultivation, cell breakdown, and the production of the raw extracts were performed as described above. Because a molecule of glucose is released with each conversion of a molecule of saccharose, the activity was determined by measuring the glucose quantity released per time unit. Ftf activities were determined over a period of 60 minutes at 30° C. using a quantity of raw extract containing approximately 1 to 3 mU fructosyl transferase in a volume of 1 ml with 100 mM Scr in 50 mM K₂HPO₄/K₂HPO₄ buffer, pH 6.5. One unit hereby corresponds to the release of 1 μmol glucose per minute in the presence of 100 mM saccharose at 30° C. and a pH value of 6.5. After 1 hour of incubation, the reaction was terminated by 10 minutes of heating at 100° C. This was followed by 10 minutes of centrifugation at 10,000×g. Then the released quantity of glucose was determined in a 100 μl aliquot using the coupled glucose oxidase (GOD)/peroxidase (POD) enzyme test (Werner et al., Z. Analyt. Chem., 252 (1970) 224-228). For this, the GOD-Perid® test (Roche Diagnostics) that photometrically detects the oxidation of the dye 2,2′-azino-di-(3-ethylbenzthioazoline sulfonate) (ABTS®) was used. The test was performed in a total volume of 1 ml with 80 μg/ml GOD, 10 μg/ml POD, and 1 μg/ml ABTS® in 50 mM KH₂PO₄/K₂HPO₄ buffer, pH value 6.5, whereby the extinction was measured at 578 nm after 30 minutes. The test was calibrated using standard glucose solutions. The expression strains found for the various raw extracts are summarized in Table 1.

TABLE 1
Specific Ftf activities in the raw extract of
E. coli strains that were transformed with the
plasmids according to the invention

| Strain, ftf gene | Induction duration | | Spec. Ftf activity in |
|---|---|---|---|
| variant | (50 ml dΥT, 30° C.) | OD₆₀₀ | raw extract [U/mg] |
| E. coli (pJOE2702) | 6 h | 5.8 | <0.1 |
| E. coli (pDHE113), | 3 h | 1.6 | 1.8 |
| ftf | 6 h | 2.0 | 0.9 |
| E. coli (pDHE132) | 3 h | 1.5 | 5.2 |
| ftf(Δ2254→2385) | 6 h | 4.9 | 7.8 |
| E. coli (pDHE132) | | | |
| ftf(Δ2254→2385) | | | |
| E. coli (pDHE225) | 3 h | 1.7 | 0.5 |
| ftf(Δ4→222) | | | |
| E. coli (pDHE225) | 6 h | 3.8 | 2.6 |
| ftf(Δ4→222) | | | |
| E. coli (pDHE172) | 3 h | 1.7 | 1.5 |
| ftf(Δ4→222, | | | |
| Δ2254→2385) | | | |
| E. coli (pDHE172) | 6 h | 5.7 | 5.0 |
| ftf(Δ4→222, | | | |
| Δ2254→2385) | | | |
| E. coli (pDHE171) | 3 h | 1.8 | 1.7 |
| lacZα(1→83)::ftf | | | |
| (105→2388) | | | |
| E. coli (pDHE171) | 6 h | 4.1 | 2.5 |
| lacZα(1→83)::ftf | | | |
| (105→2388) | | | |
| E. coli (pDHE143) | 3 h | 1.7 | 1.0 |
| lacZα(1→83Δ)::ftf | | | |
| (105→2388) | | | |
| Δ2254→2385) | | | |
| E. coli (pDHE143) | 6 h | 5.0 | 5.2 |
| lacZα(1→83Δ)::ftf | | | |
| (105→2388) | | | |
| Δ2254→2385) | | | |
| E. coli (pDHE140) | 3 h | 1.7 | 2.2 |
| lacZα(1→83)::ftf | | | |
| (105→2388) | | | |
| Δ2254→2385), in | | | |
| pBluescript KSII+ | | | |
| E. coli (pDHE140) | 6 h | 2.9 | 1.0 |
| lacZα(1→83)::ftf | | | |
| (105→2388) | | | |
| Δ2254→2385), in | | | |
| pBluescript KSII+ | | | |

As seen in the table, E. coli strains transformed with an ftf nucleotide molecule contained in vector pJOE2702 show a clearly higher cell density after an induction period of 6 hours than the E. coli strain containing the complete ftf gene, i.e. the growth of these strains is therefore clearly improved. At the same time, the volume yield of the expressed protein is also clearly higher than for the E. coli strain with the complete ftf gene.

### EXAMPLE 4

### Isolation and Immobilization of the Fructosyl Transferase

Preculture 1: 5 ml medium (per 1,000 ml water, pH 7.0, 16 g tryptone, 10 g yeast extract, 5 g NaCl, and 100 mg ampicillin) were inoculated with the E. coli strain JM109 (pDEH143) and incubated for 12 to 15 hours at 37° C. with shaking (150 rpm).

Preculture 2: 200 ml of same medium were inoculated with 1 ml of preculture 1 and incubated for 12 to 15 hours at 37° C. with shaking.

Cultivation in fermenter and expression of fructosyl transferase: 16 l of medium to which 0.2% L-rhamnose had been added for expressing the fructosyl transferase were inoculated with 200 ml preculture 2 and cultivated at 30° C., 400 rpm, and 0.5 vvm air for approximately 12 hours, i.e. until an optical density (OD₅₇₈) of approximately 0.6 or higher.

Cell harvest and breakdown: The cells were centrifuged off with a continuous centrifuge, for example a Contifuge (Heraeus) at 4° C. and 24,300×g. The cell mass was washed once with 2,000 ml of a 100 mM phosphate buffer, pH value 6.5, and then resuspended in 100 ml of phosphate buffer. Then the cells were broken down in the homogenizer at 800 bar. After this, the suspension was centrifuged at 17,360×g in order to separate cell remnants from the enzyme in the supernatant.

Immobilization: The raw extract was first freeze-dried. 23.6 g (approximately 10 g protein) of the lyophilisate were dissolved in 500 ml 1 M phosphate buffer, pH value 6.5, and incubated after addition of 100 g EUPERGIT® C (Rohm) for 94 hours at room temperature with shaking. The Ftf immobilisate was then washed with 50 mM phosphate buffer.

### EXAMPLE 5

### Conversion of Saccharose with the Ftf Protein for Polyfructan Production

60 l of a 10% saccharose solution, pH value 6.5, were incubated after addition of 640 U of fructosyl transferase per liter batch solution for 28 hours at 30° C. with stirring, whereby a raw extract of the E. coli strain JM109 (pDHE143) was used. The solution then underwent direct ultrafiltration (Sartocon Mini; 3 modules with a molecular retention capacity of 100,000 Dalton). The resulting retentate of 4.5 l was diluted twice, each time with 6 l desalinated water, and was then concentrated to 4.5 l. Then inulin was precipitated with isopropanol (final concentration 62 wt. %). The separated precipitate was washed with 5 l of the same isopropanol solution and then gently dried at 45° C. This yielded 0.36 kg of a white product with a dry substance content (DS) of 93%. In relation to the saccharose consumption, a yield of 8.5% was achieved. The molecular weight of inulin, measured using the HPLC-GPC method, is 40×10⁶ g/mol, with a degree of branching of 3 mol %.

### EXAMPLE 7

### Conversion of Ftf Inulin with Endo-Inulinase for Producing Fructooligosaccharides

20 g of the Ftf inulin obtained in Example 6 with a dry substance of 95% were incubated, after preparing a 1% solution by heating for 15 minutes at 95% and adjustment of the pH value to 5.0 by using 0.1 molar acetic acid, with endo-inulinase (0.13 ml/batch; SP 168, Novo) for 8 hours at 50° C. with stirring. After inactivation of the enzyme by heating for 15 minutes at 95%, the fructooligosaccharides formed were separated using ultrafiltration (Sartocon Mini, module with a molecular retention capacity of 10,000 Dalton). The permeate contained 7.5 g of fructooligosaccharides, while the retentate diluted to 1 l accordingly contained 11.6 g of dry substance. The retentate was incubated, after readjusting the pH value, with endo-inulinase at a constant enzyme/substrate ratio, as described above. This process was repeated for a total of five times. The following chain length distribution was determined in the combined permeates using gel permeation chromatography:

| | |
|---|---|
| DP 1 | 9.5% |
| DP 2-5 | 50% |
| DP 6-10 | 18.0% |
| DP 11-25 | 23.5% |
| Total: | 100% |

The carbohydrate composition was determined as follows: 0.5 ml of combined permeate solution (1% dry substance) were hydrolyzed after addition of 0.5 ml of 1% oxalic acid for 2.5 h at 65° C. An HPAEC analysis showed that fructose was the only carbohydrate component, i.e. the isolated oligosaccharides were type Fₙ homooligomeric fructooligosaccharides with n=1-25.

### EXAMPLE 8

### Conversion of Saccharose with the Ftf Protein and Immobilized Endo-Inulinase

Endo-inulinase (for example, SP 168; NOVO) was immobilized as described in Example 5 for fructosyl transferase on EUPERGIT® C(Röhm). 50 g of immobilized fructosyl transferase were added to 1 l of a 10% saccharose solution, pH value 6.5, at 30° C. (see Example 2), and 20 g of immobilized endo-inulinase were added and incubated with slow stirring. Samples were removed hourly and tested for saccharose content. As soon as no saccharose could be detected any longer, the reaction was terminated, and the enzymes were removed from the batch by filtration. By using gel permeation chromatography, the composition of the resulting product solution was determined as follows:

| | |
|---|---|
| DP 1 | 32.5% |
| DP 2-5 | 35.0% |
| DP 6-10 | 14.0% |
| DP 11-25 | 18.5% |
| Total: | 100% |

This means that products with a substantially higher fructose content were obtained than in Example 7.

### EXAMPLE 9

### Production of Hydrogenated Fructooligosaccharides

The homooligomeric fructooligosaccharides with a chain length from DP 1-DP 25 obtained in Examples 7 and 8 each were adjusted by evaporation to a dry substance content of 10%. 450 ml of each solution were hydrogenated in a laboratory autoclave in the presence of Raney nickel for 10 hours with hydrogen at 150 bar and 80° C. The resulting solution was pumped from the autoclave, filtered, and purified with ion exchangers. An analysis showed that the solution contained (fructosyl)ₙ mannitol, (fructosyl)ₙ sorbitol (n=1-24), as well as mannitol and sorbitol that had been produced from the fructose in the starting solution. Mannitol and sorbitol were separated with the help of known chromatography methods so that a product consisting of (fructosyl)ₙ mannitol and (fructosyl)ₙ sorbitol was obtained.

### EXAMPLE 10

### Conversion of Saccharose with Fructosyl Transferase Endo-Inulinase and Arthrobacter ureafaciens for Producing Difructose Dianhydride III

Cells of the Arthrobacter ureafaciens strain ATCC 21124 were inoculated in four shaking flasks with, in each case, 100 ml of a nutrient solution containing 8 g chicory inulin (Raftiline®), 2 g Na₂HPO₄, 1 g KH₂PO₄, 1 g NH₄NO₃, 0.5 g MgSO₄, 0.01 g FeSO₄, 0.03 g CaSO₄, 0.5 g yeast extract in desalinated water. Then a 24-hour incubation was performed at 27° C. with shaking at 150 rpm. After this, the cells were harvested by centrifugation and were immobilized in polyvinyl alcohol gel particles.

At 30° C., 50 mg of immobilized fructosyl transferase (see Example 5) and 20 g of immobilized endo-inulinase as well as immobilized A. ureafaciens cells were added to 1 l of a 10% saccharose solution, pH value 6.5. After this, incubation was performed at 30° C. with slow stirring. Samples were removed hourly and tested for their saccharose content. As soon as no saccharose could be detected any longer, the reaction was terminated and the enzymes were separated from the batch by filtration.

An HPLC analysis of the resulting product solution showed the production of 18 g DFA III.

1. Nucleic acid molecule with a nucleic acid sequence, shown in SEQ ID No. 1, or a nucleic acid sequence encoding the amino acid sequence shown in SEQ ID No. 2, whereby the nucleic acid sequence encodes a polypeptide with the activity of a fructosyl transferase and has at least one deletion in the 5′ or 3′ region in the nucleic acid sequence, whereby the deletion is selected from the group consisting of:
a) Deletion of nucleotides 4 to 222,
b) Deletion of nucleotides 1 to 104, and
c) Deletion of nucleotides 2254 to 2385.

2. Nucleic acid molecule according to claim 1, whereby the nucleic acid sequence is selected from the group consisting of:
a) a nucleic acid molecule with a nucleic acid sequence shown in SEQ ID No. 3, or a complementary strand thereof;
b) a nucleic acid molecule encoding an amino acid sequence shown in SEQ ID No. 4, or a complementary strand thereof;
c) a nucleic acid molecule with a nucleic acid sequence shown in SEQ ID No. 7, or a complementary strand thereof;
d) a nucleic acid molecule encoding an amino acid sequence shown in SEQ ID No. 8, or a complementary strand thereof;
e) a nucleic acid molecule with a nucleic acid sequence shown in SEQ ID No. 9, or a complementary strand thereof;
f) a nucleic acid molecule encoding an amino acid sequence shown in SEQ ID No. 10, or a complementary strand thereof;
g) a nucleic acid molecule that can be obtained by substitution, addition, inversion, and/or deletion of one or more bases of a nucleic acid molecule according to a) to f), or a complementary strand thereof.

3. Nucleic acid molecule according to claim 1 or 2, whereby the sequences deleted in the 5′ region of the nucleic acid sequence of the ftf gene are substituted with at least one region of another gene.

4. Nucleic acid molecule according to claim 3, whereby the sequences deleted in the 5′ region of the nucleic acid sequence of the ftf gene are substituted with sequences of the lacZα gene, whereby the nucleic acid molecule is selected from the group consisting of:
a) a nucleic acid molecule with a nucleic acid sequence shown in SEQ ID No. 5, or a complementary strand thereof;
b) a nucleic acid molecule encoding an amino acid sequence shown in SEQ ID No. 6, or a complementary strand thereof;
c) a nucleic acid molecule with a nucleic acid sequence shown in SEQ ID No. 11, or a complementary strand thereof;
d) a nucleic acid molecule encoding an amino acid sequence shown in SEQ ID No. 12, or a complementary strand thereof; and
e) a nucleic acid molecule that can be obtained by substitution, addition, inversion, and/or deletion of one or more bases of a nucleic acid molecule according to a) to d), or a complementary strand thereof.

5. Nucleic acid molecule according to one of claims 1 to 4, which is a DNA or RNA molecule.

6. Vector, comprising at least one nucleic acid molecule according to one of claims 1 to 5.

7. Vector according to claim 6, whereby the vector is a plasmid, cosmid, bacteriophage, liposome, or virus.

8. Vector according to claim 6 or 7, whereby the at least one nucleic acid molecule is under the functional control of at least one regulatory element, which in prokaryotic and eukaryotic cells ensures the transcription of a translatable RNA and/or the translation of the RNA into a polypeptide or protein.

9. Vector according to claim 8, whereby the at least one regulator element is a promoter, enhancer, silencer, or 3′ transcription terminator.

10. Vector according to claim 8 or 9, whereby the at least one regulatory element is a signal sequence for localizing the polypeptide or protein encoded by the nucleic acid molecule within certain cell organelles, compartments, or in the extracellular space.

11. Vector according to one of claims 8 to 10, whereby the at least one regulatory element stems from the L-rhamnose operon of Escherichia coli.

12. Host cell, containing at least one nucleic acid molecule according to one of claims 1 to 5 or at least one vector according to one of claims 6 to 11.

13. Host cell according to claim 12, whereby the host cell is a prokaryotic or eukaryotic cell.

14. Host cell according to claim 12 or 13, selected from bacteria, yeast cells, and plant cells.

15. Host cell according to claim 14, which is a potato, topinambur, artichoke, chicory, manioc, or sugar beet cell.

16. Plant that contains in at least one of its cells at least one nucleic acid molecule according to one of claims 1 to 5 or at least one vector according to claims 6 to 10 or the at least one cell according to claim 15.

17. Plant according to claim 16, which is a potato, topinambur, artichoke, chicory, manioc, or sugar beet plant.

18. Seeds, fruit, reproduction material, harvest material, and plant tissue that can be obtained from a plant according to claim 16 or 17.

19. Protein with the activity of a fructosyl transferase, where said protein catalyzes the conversion of saccharose into a polyfructan with primarily β-2,1 bonds and can be obtained by expression of a nucleic acid molecule according to one of claims 1 to 5.

20. Protein according to claim 19, which can be obtained by expression in a host cell according to one of claims 12 to 15 or in a plant according to claim 16 or 17.

21. Antibody that specifically identifies and binds a protein according to claim 19 or 20.

22. Antibody according to claim 21, which is a monoclonal, polyclonal, and/or modified antibody.

23. Antibody which is directed against an antibody according to claim 21 or 22.

24. Method for producing a plant that is modified by genetic engineering and produces a polyfructan, comprising:
a) the transformation of one or several plant cells with a vector according to one of claims 6 to 10,
b) the integration of the fructosyl transferase gene contained in the vector into the genome of the transformed cell(s), and
c) the regeneration of intact plants that produce a polyfructan.

25. Method for producing a polyfructan, whereby host cells according to claim 15 or plants according to claim 16 or 17 or plant tissue according to claim 18 is cultivated under conditions suitable for the production of the polyfructan, and the polyfructan is isolated, and purified.

26. Method for producing a polyfructan, comprising the following steps:
a) Cultivation and multiplication of a host cell according to one of claims 12 to 15 in a suitable nutrient medium and under suitable cultivation conditions,
b) Breakdown of the produced cell material using suitable physical, chemical, and/or enzymatic methods, and isolation and/or purification of a protein with the activity of a fructosyl transferase, either as raw extract, in highly purified form, and/or in immobilized form on a solid carrier,
c) Treatment of a saccharose solution with a protein, obtained under b), with the activity of a fructosyl transferase, and
d) Isolation and purification of the produced polyfructan from the reaction batch.

27. Polyfructan, produced according to one of the methods according to claim 25 or 26.

28. Polyfructan according to claim 27, which is inulin with a degree of polymerization >100 and a degree of branching ≦3%.

29. Method for producing fructooligosaccharides, whereby inulin according to claim 28 is treated under suitable conditions with an immobilized or non-immobilized endo-inulinase, and the produced fructooligosaccharides are then isolated from the reaction batch and purified.

30. Method for producing fructooligosaccharides, whereby a saccharose solution is treated under suitable conditions simultaneously with an immobilized or non-immobilized protein according to claim 19 or 20 and an immobilized or non-immobilized endo-inulinase, and the produced fructooligosaccharides are then isolated from the reaction batch and purified.

31. Fructooligosaccharides, produced according to the method according to claim 29 or 30.

32. Hydrogenated fructooligosaccharides, produced by hydrogenation of the fructooligosaccharides according to claim 31.

33. Method for producing difructose dianhydrides, whereby inulin according to claim 28 is treated under suitable conditions simultaneously with an immobilized or non-immobilized endo-inulinase and immobilized or non-immobilized cells of Arthrobacter globiformes or Arthrobacter ureafaciens, and the produced difructose dianhydrides are then isolated from the reaction batch and purified.

34. Method for producing difructose dianhydrides, whereby a saccharose solution is treated simultaneously with an immobilized or non-immobilized protein according to claim 19 or 20 and an immobilized or non-immobilized endo-inulinase and immobilized or non-immobilized cells of Arthrobacter globiformes or Arthrobacter ureafaciens, and the produced difructose dianhydrides are then isolated from the reaction batch and purified.

35. Difructose dianhydrides, produced according to one of the methods according to claim 33 or 34.

36. Use of inulin according to claim 28 for producing inulin ethers and inulin esters.

37. Use of the fructooligosaccharides according to claim 31 as a food additive, dietetic food, or animal feed additive.

38. Use of the hydrogenated fructooligosaccharides according to claim 31 as a food additive, dietetic food, or animal feed additive.

39. Use of the difructose dianhydrides according to claim 35 as a food additive, dietetic food, or animal feed additive.


SEQUENCE LISTING (18 sequences)

SEQ ID NO: 1 - DNA, 2388 bp, Streptococcus mutans
atggaaactaaagttagaaaaaagatgtataagaaagggaaattttgggtggtagccaccatcacgactgctatgctgactggaattgggctctcttctgttcaggcagatgaagccaattcaactcaagtttcttcagaattggctgaaagaagtcaggttcaagaaaatacaactgcttcatcatcagcagcagaaaatcaggctaagactgaagttcaagaaactccatcaacaaatcccgcagctgctactgttgagaacactgatcaaacaactaaggtgataacagataatgctgctgttgaatcaaaagcaagtaaaactaaggatcaagcagctaccgtaactaaaacagcagctagtacaccggaagtaggtcaaacaaatgaaaaggctaaggcaactaaagaagctgacataactacgccaaagaatacaatagatgaatacggcctaacagaacaggctcgtaagattgctactgaagctggtattaatttaagcagtttgacacaaaagcaagttgaagcattaaataaagttaaattaacgagtgatgctcaaacgggtcatcaaatgacctatcaagaatttgacaagattgctcaaacgttgatagctcaagatgaacgctatgctatcccttattttaatgcaaaagcaatcaaaaatatgaaggcggctacaacgcgtgatgcccaaacgggtcaaatagctgatttggatgtttgggattcttggccagttcaggatgctaagactggtgaagttattaattggaatggttatcagcttgttgttgctatgatgggcattccaaatactaatgataatcatatttatcttctttataataaatatggagataataattttgatcattggaaaaatgcaggttctatctttggttataatgaaacacccctaactcaagaatggtcaggttcagctaccgtaaatgaagatggaagtttgcagttattctacaccaaggttgatactagtgacaaaaacagtaacaatcaacgtttagcaacagcgactgtaaatcttggctttgatgaccaagatgttagaattctttctgttgaaaatgataaagttttaacgcctgaaggcggtgatggctatcattatcaaagttatcaacaatggcgttcaacctttacaggtgctgataatattgctatgcgtgatccacatgtcattgaagatgagaatggagatcgctatcttgtctttgaggctagtacaggtacagagaattatcaaggtgaagatcagatttacaactttactaactatggcggcagctctgcttataatgttaaaagtctttttagatttttagatgatcaagatatgtataaccgtgcaagctgggccaatgcagctattggtattttaaaacttaagggcgataaaaaaacacctgaggtagatcaattttacacgcctttactaagttcaacaatggtttcggatgaactcgagcgacccaatgtggttaaattaggagataagtactatctttttacagcttcacgtcttaatcacggaagtaacaacgatgcttggaataaagcaaatgaagttgttggtgataatgtcgttatgctaggttatgtttctgatcaattgactaacggctacaaacccttaaataatagtggtgtggttttaacagcttcagttccagcagattggcgaacggcgacttactcttattatgctgttccagtagctggatcatctgataccctgttaatgacggcttatatgaccaatcgtaatgaagtcgcaggtaaaggtaagaattcaacctgggcacctagttttctgattcaggttttaccagatgggactacaaaagtcttagcagaaatgacacaacagggtgattggatttgggatgaaccaagtcgcacgacagacactgttggcacgctggatacagcctatcttccaggtgaaaatgatggttatattgattggaatgttattggtggctacggtttgaagccacatacaccgggacaatatcaaccaactgttccatcaacaccaattcatacagatgacattatttcttttgaagtctcttttgatggtcatctcgttattaaacctgtcaaagtaaataatgactccgctggtcgaattgaccaatcaaggaattcaggtgggtctcttaatgttgcctttaatgtctctgcgggcggaaatatttctgtcaaaccttctcaaaaatcgattaataacacaaaagaaacaaagaaagctcatcatgtttcaacagaaaagaaacagaaaaaaggaaattctttctttgcagctttattagctcttttcagtgctttctgtgtaagcattggttttaaataa

SEQ ID NO: 2 - protein, 795 aa, Streptococcus mutans
METKVRKKMYKKGKFWVVATITTAMLTGIGLSSVQADEANSTQVSSELAERSQVQENTTASSSAAENQAKTEVQETPSTNPAAATVENTDQTTKVITDNAAVESKASKTKDQAATVTKTAASTPEVGQTNEKAKATKEADITTPKNTIDEYGLTEQARKIATEAGINLSSLTQKQVEALNKVKLTSDAQTGHQMTYQEFDKIAQTLIAQDERYAIPYFNAKAIKNMKAATTRDAQTGQIADLDVWDSWPVQDAKTGEVINWNGYQLVVAMMGIPNTNDNHIYLLYNKYGDNNFDHWKNAGSIFGYNETPLTQEWSGSATVNEDGSLQLFYTKVDTSDKNSNNQRLATATVNLGFDDQDVRILSVENDKVLTPEGGDGYHYQSYQQWRSTFTGADNIAMRDPHVIEDENGDRYLVFEASTGTENYQGEDQIYNFTNYGGSSAYNVKSLFRFLDDQDMYNRASWANAAIGILKLKGDKKTPEVDQFYTPLLSSTMVSDELERPNVVKLGDKYYLFTASRLNHGSNNDAWNKANEVVGDNVVMLGYVSDQLTNGYKPLNNSGVVLTASVPADWRTATYSYYAVPVAGSSDTLLMTAYMTNRNEVAGKGKNSTWAPSFLIQVLPDGTTKVLAEMTQQGDWIWDEPSRTTDTVGTLDTAYLPGENDGYIDWNVIGGYGLKPHTPGQYQPTVPSTPIHTDDIISFEVSFDGHLVIKPVKVNNDSAGRIDQSRNSGGSLNVAFNVSAGGNISVKPSQKSINNTKETKKAHHVSTEKKQKKGNSFFAALLALFSAFCVSIGFK

SEQ ID NO: 3 - DNA, 2169 bp, Streptococcus mutans
atggaaactccatcaacaaatcccgcagctgctactgttgagaacactgatcaaacaactaaggtgataacagataatgctgctgttgaatcaaaagcaagtaaaactaaggatcaagcagctaccgtaactaaaacagcagctagtacaccggaagtaggtcaaacaaatgaaaaggctaaggcaactaaagaagctgacataactacgccaaagaatacaatagatgaatacggcctaacagaacaggctcgtaagattgctactgaagctggtattaatttaagcagtttgacacaaaagcaagttgaagcattaaataaagttaaattaacgagtgatgctcaaacgggtcatcaaatgacctatcaagaatttgacaagattgctcaaacgttgatagctcaagatgaacgctatgctatcccttattttaatgcaaaagcaatcaaaaatatgaaggcggctacaacgcgtgatgcccaaacgggtcaaatagctgatttggatgtttgggattcttggccagttcaggatgctaagactggtgaagttattaattggaatggttatcagcttgttgttgctatgatgggcattccaaatactaatgataatcatatttatcttctttataataaatatggagataataattttgatcattggaaaaatgcaggttctatctttggttataatgaaacacccctaactcaagaatggtcaggttcagctaccgtaaatgaagatggaagtttgcagttattctacaccaaggttgatactagtgacaaaaacagtaacaatcaacgtttagcaacagcgactgtaaatcttggctttgatgaccaagatgttagaattctttctgttgaaaatgataaagttttaacgcctgaaggcggtgatggctatcattatcaaagttatcaacaatggcgttcaacctttacaggtgctgataatattgctatgcgtgatccacatgtcattgaagatgagaatggagatcgctatcttgtctttgaggctagtacaggtacagagaattatcaaggtgaagatcagatttacaactttactaactatggcggcagctctgcttataatgttaaaagtctttttagatttttagatgatcaagatatgtataaccgtgcaagctgggccaatgcagctattggtattttaaaacttaagggcgataaaaaaacacctgaggtagatcaattttacacgcctttactaagttcaacaatggtttcggatgaactcgagcgacccaatgtggttaaattaggagataagtactatctttttacagcttcacgtcttaatcacggaagtaacaacgatgcttggaataaagcaaatgaagttgttggtgataatgtcgttatgctaggttatgtttctgatcaattgactaacggctacaaacccttaaataatagtggtgtggttttaacagcttcagttccagcagattggcgaacggcgacttactcttattatgctgttccagtagctggatcatctgataccctgttaatgacggcttatatgaccaatcgtaatgaagtcgcaggtaaaggtaagaattcaacctgggcacctagttttctgattcaggttttaccagatgggactacaaaagtcttagcagaaatgacacaacagggtgattggatttgggatgaaccaagtcgcacgacagacactgttggcacgctggatacagcctatcttccaggtgaaaatgatggttatattgattggaatgttattggtggctacggtttgaagccacatacaccgggacaatatcaaccaactgttccatcaacaccaattcatacagatgacattatttcttttgaagtctcttttgatggtcatctcgttattaaacctgtcaaagtaaataatgactccgctggtcgaattgaccaatcaaggaattcaggtgggtctcttaatgttgcctttaatgtctctgcgggcggaaatatttctgtcaaaccttctcaaaaatcgattaataacacaaaagaaacaaagaaagctcatcatgtttcaacagaaaagaaacagaaaaaaggaaattctttctttgcagctttattagctcttttcagtgctttctgtgtaagcattggttttaaataa

SEQ ID NO: 4 - protein, 722 aa, Streptococcus mutans
METPSTNPAAATVENTDQTTKVITDNAAVESKASKTKDQAATVTKTAASTPEVGQTNEKAKATKEADITTPKNTIDEYGLTEQARKIATEAGINLSSLTQKQVEALNKVKLTSDAQTGHQMTYQEFDKIAQTLIAQDERYAIPYFNAKAIKNMKAATTRDAQTGQIADLDVWDSWPVQDAKTGEVINWNGYQLVVAMMGIPNTNDNHIYLLYNKYGDNNFDHWKNAGSIFGYNETPLTQEWSGSATVNEDGSLQLFYTKVDTSDKNSNNQRLATATVNLGFDDQDVRILSVENDKVLTPEGGDGYHYQSYQQWRSTFTGADNIAMRDPHVIEDENGDRYLVFEASTGTENYQGEDQIYNFTNYGGSSAYNVKSLFRFLDDQDMYNRASWANAAIGILKLKGDKKTPEVDQFYTPLLSSTMVSDELERPNVVKLGDKYYLFTASRLNHGSNNDAWNKANEVVGDNVVMLGYVSDQLTNGYKPLNNSGVVLTASVPADWRTATYSYYAVPVAGSSDTLLMTAYMTNRNEVAGKGKNSTWAPSFLIQVLPDGTTKVLAEMTQQGDWIWDEPSRTTDTVGTLDTAYLPGENDGYIDWNVIGGYGLKPHTPGQYQPTVPSTPIHTDDIISFEVSFDGHLVIKPVKVNNDSAGRIDQSRNSGGSLNVAFNVSAGGNISVKPSQKSINNTKETKKAHHVSTEKKQKKGNSFFAALLALFSAFCVSIGFK

SEQ ID NO: 5 - DNA, 2367 bp, Streptococcus mutans
atgaccatgattacgccaagcgcgcaattaaccctcactaaagggaacaaaagctgggtaccgggccccccctcgaggtcgacggcagatgaagccaattcaactcaagtttcttcagaattggctgaaagaagtcaggttcaagaaaatacaactgcttcatcatcagcagcagaaaatcaggctaagactgaagttcaagaaactccatcaacaaatcccgcagctgctactgttgagaacactgatcaaacaactaaggtgataacagataatgctgctgttgaatcaaaagcaagtaaaactaaggatcaagcagctaccgtaactaaaacagcagctagtacaccggaagtaggtcaaacaaatgaaaaggctaaggcaactaaagaagctgacataactacgccaaagaatacaatagatgaatacggcctaacagaacaggctcgtaagattgctactgaagctggtattaatttaagcagtttgacacaaaagcaagttgaagcattaaataaagttaaattaacgagtgatgctcaaacgggtcatcaaatgacctatcaagaatttgacaagattgctcaaacgttgatagctcaagatgaacgctatgctatcccttattttaatgcaaaagcaatcaaaaatatgaaggcggctacaacgcgtgatgcccaaacgggtcaaatagctgatttggatgtttgggattcttggccagttcaggatgctaagactggtgaagttattaattggaatggttatcagcttgttgttgctatgatgggcattccaaatactaatgataatcatatttatcttctttataataaatatggagataataattttgatcattggaaaaatgcaggttctatctttggttataatgaaacacccctaactcaagaatggtcaggttcagctaccgtaaatgaagatggaagtttgcagttattctacaccaaggttgatactagtgacaaaaacagtaacaatcaacgtttagcaacagcgactgtaaatcttggctttgatgaccaagatgttagaattctttctgttgaaaatgataaagttttaacgcctgaaggcggtgatggctatcattatcaaagttatcaacaatggcgttcaacctttacaggtgctgataatattgctatgcgtgatccacatgtcattgaagatgagaatggagatcgctatcttgtctttgaggctagtacaggtacagagaattatcaaggtgaagatcagatttacaactttactaactatggcggcagctctgcttataatgttaaaagtctttttagatttttagatgatcaagatatgtataaccgtgcaagctgggccaatgcagctattggtattttaaaacttaagggcgataaaaaaacacctgaggtagatcaattttacacgcctttactaagttcaacaatggtttcggatgaactcgagcgacccaatgtggttaaattaggagataagtactatctttttacagcttcacgtcttaatcacggaagtaacaacgatgcttggaataaagcaaatgaagttgttggtgataatgtcgttatgctaggttatgtttctgatcaattgactaacggctacaaacccttaaataatagtggtgtggttttaacagcttcagttccagcagattggcgaacggcgacttactcttattatgctgttccagtagctggatcatctgataccctgttaatgacggcttatatgaccaatcgtaatgaagtcgcaggtaaaggtaagaattcaacctgggcacctagttttctgattcaggttttaccagatgggactacaaaagtcttagcagaaatgacacaacagggtgattggatttgggatgaaccaagtcgcacgacagacactgttggcacgctggatacagcctatcttccaggtgaaaatgatggttatattgattggaatgttattggtggctacggtttgaagccacatacaccgggacaatatcaaccaactgttccatcaacaccaattcatacagatgacattatttcttttgaagtctcttttgatggtcatctcgttattaaacctgtcaaagtaaataatgactccgctggtcgaattgaccaatcaaggaattcaggtgggtctcttaatgttgcctttaatgtctctgcgggcggaaatatttctgtcaaaccttctcaaaaatcgattaataacacaaaagaaacaaagaaagctcatcatgtttcaacagaaaagaaacagaaaaaaggaaattctttctttgcagctttattagctcttttcagtgctttctgtgtaagcattggttttaaataa

SEQ ID NO: 6 - protein, 788 aa, Streptococcus mutans
MTMITPSAQLTLTKGNKSWVPGPPSRSTADEANSTQVSSELAERSQVQENTTASSSAAENQAKTEVQETPSTNPAAATVENTDQTTKVITDNAAVESKASKTKDQAATVTKTAASTPEVGQTNEKAKATKEADITTPKNTIDEYGLTEQARKIATEAGINLSSLTQKQVEALNKVKLTSDAQTGHQMTYQEFDKIAQTLIAQDERYAIPYFNAKAIKNMKAATTRDAQTGQIADLDVWDSWPVQDAKTGEVINWNGYQLVVAMMGIPNTNDNHIYLLYNKYGDNNFDHWKNAGSIFGYNETPLTQEWSGSATVNEDGSLQLFYTKVDTSDKNSNNQRLATATVNLGFDDQDVRILSVENDKVLTPEGGDGYHYQSYQQWRSTFTGADNIAMRDPHVIEDENGDRYLVFEASTGTENYQGEDQIYNFTNYGGSSAYNVKSLFRFLDDQDMYNRASWANAAIGILKLKGDKKTPEVDQFYTPLLSSTMVSDELERPNVVKLGDKYYLFTASRLNHGSNNDAWNKANEVVGDNVVMLGYVSDQLTNGYKPLNNSGVVLTASVPADWRTATYSYYAVPVAGSSDTLLMTAYMTNRNEVAGKGKNSTWAPSFLIQVLPDGTTKVLAEMTQQGDWIWDEPSRTTDTVGTLDTAYLPGENDGYIDWNVIGGYGLKPHTPGQYQPTVPSTPIHTDDIISFEVSFDGHLVIKPVKVNNDSAGRIDQSRNSGGSLNVAFNVSAGGNISVKPSQKSINNTKETKKAHHVSTEKKQKKGNSFFAALLALFSAFCVSIGFK

SEQ ID NO: 7 - DNA, 2256 bp, Streptococcus mutans
atggaaactaaagttagaaaaaagatgtataagaaagggaaattttgggtggtagccaccatcacgactgctatgctgactggaattgggctctcttctgttcaggcagatgaagccaattcaactcaagtttcttcagaattggctgaaagaagtcaggttcaagaaaatacaactgcttcatcatcagcagcagaaaatcaggctaagactgaagttcaagaaactccatcaacaaatcccgcagctgctactgttgagaacactgatcaaacaactaaggtgataacagataatgctgctgttgaatcaaaagcaagtaaaactaaggatcaagcagctaccgtaactaaaacagcagctagtacaccggaagtaggtcaaacaaatgaaaaggctaaggcaactaaagaagctgacataactacgccaaagaatacaatagatgaatacggcctaacagaacaggctcgtaagattgctactgaagctggtattaatttaagcagtttgacacaaaagcaagttgaagcattaaataaagttaaattaacgagtgatgctcaaacgggtcatcaaatgacctatcaagaatttgacaagattgctcaaacgttgatagctcaagatgaacgctatgctatcccttattttaatgcaaaagcaatcaaaaatatgaaggcggctacaacgcgtgatgcccaaacgggtcaaatagctgatttggatgtttgggattcttggccagttcaggatgctaagactggtgaagttattaattggaatggttatcagcttgttgttgctatgatgggcattccaaatactaatgataatcatatttatcttctttataataaatatggagataataattttgatcattggaaaaatgcaggttctatctttggttataatgaaacacccctaactcaagaatggtcaggttcagctaccgtaaatgaagatggaagtttgcagttattctacaccaaggttgatactagtgacaaaaacagtaacaatcaacgtttagcaacagcgactgtaaatcttggctttgatgaccaagatgttagaattctttctgttgaaaatgataaagttttaacgcctgaaggcggtgatggctatcattatcaaagttatcaacaatggcgttcaacctttacaggtgctgataatattgctatgcgtgatccacatgtcattgaagatgagaatggagatcgctatcttgtctttgaggctagtacaggtacagagaattatcaaggtgaagatcagatttacaactttactaactatggcggcagctctgcttataatgttaaaagtctttttagatttttagatgatcaagatatgtataaccgtgcaagctgggccaatgcagctattggtattttaaaacttaagggcgataaaaaaacacctgaggtagatcaattttacacgcctttactaagttcaacaatggtttcggatgaactcgagcgacccaatgtggttaaattaggagataagtactatctttttacagcttcacgtcttaatcacggaagtaacaacgatgcttggaataaagcaaatgaagttgttggtgataatgtcgttatgctaggttatgtttctgatcaattgactaacggctacaaacccttaaataatagtggtgtggttttaacagcttcagttccagcagattggcgaacggcgacttactcttattatgctgttccagtagctggatcatctgataccctgttaatgacggcttatatgaccaatcgtaatgaagtcgcaggtaaaggtaagaattcaacctgggcacctagttttctgattcaggttttaccagatgggactacaaaagtcttagcagaaatgacacaacagggtgattggatttgggatgaaccaagtcgcacgacagacactgttggcacgctggatacagcctatcttccaggtgaaaatgatggttatattgattggaatgttattggtggctacggtttgaagccacatacaccgggacaatatcaaccaactgttccatcaacaccaattcatacagatgacattatttcttttgaagtctcttttgatggtcatctcgttattaaacctgtcaaagtaaataatgactccgctggtcgaattgaccaatcaaggaattcaggtgggtctcttaatgttgcctttaatgtctctgcgggcggaaatatttctgtcaaaccttctcaaaaataa

SEQ ID NO: 8 - protein, 751 aa, Streptococcus mutans
METKVRKKMYKKGKFWVVATITTAMLTGIGLSSVQADEANSTQVSSELAERSQVQENTTASSSAAENQAKTEVQETPSTNPAAATVENTDQTTKVITDNAAVESKASKTKDQAATVTKTAASTPEVGQTNEKAKATKEADITTPKNTIDEYGLTEQARKIATEAGINLSSLTQKQVEALNKVKLTSDAQTGHQMTYQEFDKIAQTLIAQDERYAIPYFNAKAIKNMKAATTRDAQTGQIADLDVWDSWPVQDAKTGEVINWNGYQLVVAMMGIPNTNDNHIYLLYNKYGDNNFDHWKNAGSIFGYNETPLTQEWSGSATVNEDGSLQLFYTKVDTSDKNSNNQRLATATVNLGFDDQDVRILSVENDKVLTPEGGDGYHYQSYQQWRSTFTGADNIAMRDPHVIEDENGDRYLVFEASTGTENYQGEDQIYNFTNYGGSSAYNVKSLFRFLDDQDMYNRASWANAAIGILKLKGDKKTPEVDQFYTPLLSSTMVSDELERPNVVKLGDKYYLFTASRLNHGSNNDAWNKANEVVGDNVVMLGYVSDQLTNGYKPLNNSGVVLTASVPADWRTATYSYYAVPVAGSSDTLLMTAYMTNRNEVAGKGKNSTWAPSFLIQVLPDGTTKVLAEMTQQGDWIWDEPSRTTDTVGTLDTAYLPGENDGYIDWNVIGGYGLKPHTPGQYQPTVPSTPIHTDDIISFEVSFDGHLVIKPVKVNNDSAGRIDQSRNSGGSLNVAFNVSAGGNISVKPSQK

SEQ ID NO: 9 - DNA, 2037 bp, Streptococcus mutans
atggaaactccatcaacaaatcccgcagctgctactgttgagaacactgatcaaacaactaaggtgataacagataatgctgctgttgaatcaaaagcaagtaaaactaaggatcaagcagctaccgtaactaaaacagcagctagtacaccggaagtaggtcaaacaaatgaaaaggctaaggcaactaaagaagctgacataactacgccaaagaatacaatagatgaatacggcctaacagaacaggctcgtaagattgctactgaagctggtattaatttaagcagtttgacacaaaagcaagttgaagcattaaataaagttaaattaacgagtgatgctcaaacgggtcatcaaatgacctatcaagaatttgacaagattgctcaaacgttgatagctcaagatgaacgctatgctatcccttattttaatgcaaaagcaatcaaaaatatgaaggcggctacaacgcgtgatgcccaaacgggtcaaatagctgatttggatgtttgggattcttggccagttcaggatgctaagactggtgaagttattaattggaatggttatcagcttgttgttgctatgatgggcattccaaatactaatgataatcatatttatcttctttataataaatatggagataataattttgatcattggaaaaatgcaggttctatctttggttataatgaaacacccctaactcaagaatggtcaggttcagctaccgtaaatgaagatggaagtttgcagttattctacaccaaggttgatactagtgacaaaaacagtaacaatcaacgtttagcaacagcgactgtaaatcttggctttgatgaccaagatgttagaattctttctgttgaaaatgataaagttttaacgcctgaaggcggtgatggctatcattatcaaagttatcaacaatggcgttcaacctttacaggtgctgataatattgctatgcgtgatccacatgtcattgaagatgagaatggagatcgctatcttgtctttgaggctagtacaggtacagagaattatcaaggtgaagatcagatttacaactttactaactatggcggcagctctgcttataatgttaaaagtctttttagatttttagatgatcaagatatgtataaccgtgcaagctgggccaatgcagctattggtattttaaaacttaagggcgataaaaaaacacctgaggtagatcaattttacacgcctttactaagttcaacaatggtttcggatgaactcgagcgacccaatgtggttaaattaggagataagtactatctttttacagcttcacgtcttaatcacggaagtaacaacgatgcttggaataaagcaaatgaagttgttggtgataatgtcgttatgctaggttatgtttctgatcaattgactaacggctacaaacccttaaataatagtggtgtggttttaacagcttcagttccagcagattggcgaacggcgacttactcttattatgctgttccagtagctggatcatctgataccctgttaatgacggcttatatgaccaatcgtaatgaagtcgcaggtaaaggtaagaattcaacctgggcacctagttttctgattcaggttttaccagatgggactacaaaagtcttagcagaaatgacacaacagggtgattggatttgggatgaaccaagtcgcacgacagacactgttggcacgctggatacagcctatcttccaggtgaaaatgatggttatattgattggaatgttattggtggctacggtttgaagccacatacaccgggacaatatcaaccaactgttccatcaacaccaattcatacagatgacattatttcttttgaagtctcttttgatggtcatctcgttattaaacctgtcaaagtaaataatgactccgctggtcgaattgaccaatcaaggaattcaggtgggtctcttaatgttgcctttaatgtctctgcgggcggaaatatttctgtcaaaccttctcaaaaataa

SEQ ID NO: 10 - protein, 678 aa, Streptococcus mutans
METPSTNPAAATVENTDQTTKVITDNAAVESKASKTKDQAATVTKTAASTPEVGQTNEKAKATKEADITTPKNTIDEYGLTEQARKIATEAGINLSSLTQKQVEALNKVKLTSDAQTGHQMTYQEFDKIAQTLIAQDERYAIPYFNAKAIKNMKAATTRDAQTGQIADLDVWDSWPVQDAKTGEVINWNGYQLVVAMMGIPNTNDNHIYLLYNKYGDNNFDHWKNAGSIFGYNETPLTQEWSGSATVNEDGSLQLFYTKVDTSDKNSNNQRLATATVNLGFDDQDVRILSVENDKVLTPEGGDGYHYQSYQQWRSTFTGADNIAMRDPHVIEDENGDRYLVFEASTGTENYQGEDQIYNFTNYGGSSAYNVKSLFRFLDDQDMYNRASWANAAIGILKLKGDKKTPEVDQFYTPLLSSTMVSDELERPNVVKLGDKYYLFTASRLNHGSNNDAWNKANEVVGDNVVMLGYVSDQLTNGYKPLNNSGVVLTASVPADWRTATYSYYAVPVAGSSDTLLMTAYMTNRNEVAGKGKNSTWAPSFLIQVLPDGTTKVLAEMTQQGDWIWDEPSRTTDTVGTLDTAYLPGENDGYIDWNVIGGYGLKPHTPGQYQPTVPSTPIHTDDIISFEVSFDGHLVIKPVKVNNDSAGRIDQSRNSGGSLNVAFNVSAGGNISVKPSQK

SEQ ID NO: 11 - DNA, 2235 bp, Streptococcus mutans
atgaccatgattacgccaagcgcgcaattaaccctcactaaagggaacaaaagctgggtaccgggccccccctcgaggtcgacggcagatgaagccaattcaactcaagtttcttcagaattggctgaaagaagtcaggttcaagaaaatacaactgcttcatcatcagcagcagaaaatcaggctaagactgaagttcaagaaactccatcaacaaatcccgcagctgctactgttgagaacactgatcaaacaactaaggtgataacagataatgctgctgttgaatcaaaagcaagtaaaactaaggatcaagcagctaccgtaactaaaacagcagctagtacaccggaagtaggtcaaacaaatgaaaaggctaaggcaactaaagaagctgacataactacgccaaagaatacaatagatgaatacggcctaacagaacaggctcgtaagattgctactgaagctggtattaatttaagcagtttgacacaaaagcaagttgaagcattaaataaagttaaattaacgagtgatgctcaaacgggtcatcaaatgacctatcaagaatttgacaagattgctcaaacgttgatagctcaagatgaacgctatgctatcccttattttaatgcaaaagcaatcaaaaatatgaaggcggctacaacgcgtgatgcccaaacgggtcaaatagctgatttggatgtttgggattcttggccagttcaggatgctaagactggtgaagttattaattggaatggttatcagcttgttgttgctatgatgggcattccaaatactaatgataatcatatttatcttctttataataaatatggagataataattttgatcattggaaaaatgcaggttctatctttggttataatgaaacacccctaactcaagaatggtcaggttcagctaccgtaaatgaagatggaagtttgcagttattctacaccaaggttgatactagtgacaaaaacagtaacaatcaacgtttagcaacagcgactgtaaatcttggctttgatgaccaagatgttagaattctttctgttgaaaatgataaagttttaacgcctgaaggcggtgatggctatcattatcaaagttatcaacaatggcgttcaacctttacaggtgctgataatattgctatgcgtgatccacatgtcattgaagatgagaatggagatcgctatcttgtctttgaggctagtacaggtacagagaattatcaaggtgaagatcagatttacaactttactaactatggcggcagctctgcttataatgttaaaagtctttttagatttttagatgatcaagatatgtataaccgtgcaagctgggccaatgcagctattggtattttaaaacttaagggcgataaaaaaacacctgaggtagatcaattttacacgcctttactaagttcaacaatggtttcggatgaactcgagcgacccaatgtggttaaattaggagataagtactatctttttacagcttcacgtcttaatcacggaagtaacaacgatgcttggaataaagcaaatgaagttgttggtgataatgtcgttatgctaggttatgtttctgatcaattgactaacggctacaaacccttaaataatagtggtgtggttttaacagcttcagttccagcagattggcgaacggcgacttactcttattatgctgttccagtagctggatcatctgataccctgttaatgacggcttatatgaccaatcgtaatgaagtcgcaggtaaaggtaagaattcaacctgggcacctagttttctgattcaggttttaccagatgggactacaaaagtcttagcagaaatgacacaacagggtgattggatttgggatgaaccaagtcgcacgacagacactgttggcacgctggatacagcctatcttccaggtgaaaatgatggttatattgattggaatgttattggtggctacggtttgaagccacatacaccgggacaatatcaaccaactgttccatcaacaccaattcatacagatgacattatttcttttgaagtctcttttgatggtcatctcgttattaaacctgtcaaagtaaataatgactccgctggtcgaattgaccaatcaaggaattcaggtgggtctcttaatgttgcctttaatgtctctgcgggcggaaatatttctgtcaaaccttctcaaaaataa

SEQ ID NO: 12 - protein, 744 aa, Streptococcus mutans
MTMITPSAQLTLTKGNKSWVPGPPSRSTADEANSTQVSSELAERSQVQENTTASSSAAENQAKTEVQETPSTNPAAATVENTDQTTKVITDNAAVESKASKTKDQAATVTKTAASTPEVGQTNEKAKATKEADITTPKNTIDEYGLTEQARKIATEAGINLSSLTQKQVEALNKVKLTSDAQTGHQMTYQEFDKIAQTLIAQDERYAIPYFNAKAIKNMKAATTRDAQTGQIADLDVWDSWPVQDAKTGEVINWNGYQLVVAMMGIPNTNDNHIYLLYNKYGDNNFDHWKNAGSIFGYNETPLTQEWSGSATVNEDGSLQLFYTKVDTSDKNSNNQRLATATVNLGFDDQDVRILSVENDKVLTPEGGDGYHYQSYQQWRSTFTGADNIAMRDPHVIEDENGDRYLVFEASTGTENYQGEDQIYNFTNYGGSSAYNVKSLFRFLDDQDMYNRASWANAAIGILKLKGDKKTPEVDQFYTPLLSSTMVSDELERPNVVKLGDKYYLFTASRLNHGSNNDAWNKANEVVGDNVVMLGYVSDQLTNGYKPLNNSGVVLTASVPADWRTATYSYYAVPVAGSSDTLLMTAYMTNRNEVAGKGKNSTWAPSFLIQVLPDGTTKVLAEMTQQGDWIWDEPSRTTDTVGTLDTAYLPGENDGYIDWNVIGGYGLKPHTPGQYQPTVPSTPIHTDDIISFEVSFDGHLVIKPVKVNNDSAGRIDQSRNSGGSLNVAFNVSAGGNISVKPSQK

SEQ ID NO: 13 - DNA, 27 bp, Streptococcus mutans
tatatatcatatggaaactaaagttag

SEQ ID NO: 14 - DNA, 26 bp, Streptococcus mutans
taggatccttatttaaaaccaatgct

SEQ ID NO: 15 - DNA, 35 bp, Streptococcus mutans
tatatatcatatggaaactccatcaacaaatcccg

SEQ ID NO: 16 - DNA, 33 bp, Streptococcus mutans
atatatgtcgacggcagatgaagccaattcaac

SEQ ID NO: 17 - DNA, 31 bp, Streptococcus mutans
tatatatcatatgaccatgattacgccaagc

SEQ ID NO: 18 - DNA, 31 bp, Streptococcus mutans
ttggatccttatttttgagaaggtttgacag